(19) United States
(12) Patent Application Publication
(10) Pub. No.: US 2015/0297694 A1
(43) Pub. Date: Oct. 22, 2015

(54) CANCER DIAGNOSTIC AND THERAPEUTIC METHOD TARGETING MOLECULES EXPRESSED IN CANCER STEM CELLS

(71) Applicants: NIIGATA UNIVERSITY, Niigata-shi, Niigata (JP); OTSUKA PHARMACEUTICAL CO., LTD., Chiyoda-ku, Tokyo (JP)

(72) Inventors: Hiroshi KAGAMU, Niigata-shi (JP); Ichiei NARITA, Niigata-shi (JP); Yoshihiro GOTO, Osaka-shi (JP); Takashi HAYASHI, Osaka-shi (JP)

(73) Assignees: OTSUKA PHARMACEUTICAL CO., LTD., Chiyoda-ku, Tokyo (JP); NIIGATA UNIVERSITY, Niigata-shi, Niigata (JP)

(21) Appl. No.: 14/423,929

(22) PCT Filed: Sep. 3, 2013
(86) PCT No.: PCT/JP2013/074172

§ 371 Date: Feb. 25, 2015

(30) Foreign Application Priority Data
Sep. 4, 2012 (JP) 2012-193757

(51) Int. Cl. A61K 39/00; A61K 31/17; C12N 9/14; A61K 31/551; A61K 31/4412; A61K 31/404; G01N 33/574; A61K 31/4184

(52) U.S. Cl. CPC A61K 39/0011; G01N 33/57484; A61K 31/17; A61K 31/4184; A61K 31/551; A61K 31/4412; A61K 31/404; C12N 9/14; G01N 2333/914; G01N 2333/57; G01N 2333/5406; G01N 2333/54; C12Y 306/04013

(57) ABSTRACT

The present invention provides a novel method for determining cancer malignancy, a novel cancer diagnostic method, a novel method for determining prognosis, a novel vaccine for cancer treatment, and a novel vaccine for suppressing cancer metastasis. Specifically, the invention provides a cancer malignancy evaluation method that comprises the step of measuring a DDX3X expression level in a cancer tissue, and the step of evaluating malignancy of the cancer tissue by using the DDX3X expression level.

### TECHNICAL FIELD

The present invention relates to a cancer diagnostic and therapeutic method targeting molecules expressed in cancer stem cells, particularly to a method for determining cancer malignancy, a cancer prognosis evaluation method, cancer antigenic peptides, a method for producing a cell composition for adoptive immunity, and a cancer preventing, cancer treating, cancer metastasis suppressing, or cancer recurrence suppressing agent.

### BACKGROUND ART

There has been increasing evidence that most solid malignancies consist of heterogeneous tumor cells and that a relatively small subpopulation exhibits unique characteristics, including high tumorgenicity, growth as non-adherent spheres, unlimited self-renewal, and asymmetric differentiation. The members of this unique subpopulation are referred to as cancer stem cells (CSC) because they share biologic, biochemical, and molecular features with normal stem cells. In the classical CSC model, the hierarchy between the CSC subpopulation and the relatively differentiated, bulk cancer population is rigid and one-directional. However, recent data suggests that CSC and differentiated cancer cells can convert in both directions under regulated equilibrium (Non-Patent Literature 1).

There are reports that only a few cancer cells surviving after cytotoxic chemotherapy and molecular-targeting treatment have been shown to uniformly express CD133, one of the putative CSC markers (Non-Patent Literatures 2 and 3). Because CSC possess multiple mechanisms to resist cell death—such as an altered chromatin state, and an excess of multidrug efflux transporters, anti-apoptotic factors, DNA repair gene products, and stem cell-specific growth signaling—CSC can survive under potentially lethal stresses such as cytotoxic anticancer drug, molecular-targeting therapeutic agent, and radiation therapy. These unique cancer subpopulations surviving under such potentially lethal stresses can give rise to a permanent, drug-tolerant cell population that has genetic mutations and serve as mother cells (Non-Patent Literature 2). Thus, the CSC system is most likely responsible for the majority of treatment failures and cancer recurrences. Unless an effective treatment to eradicate the CSC subpopulation is developed, it will be extremely difficult to achieve a lasting cure. Accordingly, there is a need for an effective treatment for eradicating the CSC subpopulation.

### CITATION LIST

### Non-Patent Literature

NPL 1: Li Y, Laterra J. Cancer stem cells: distinct entities or dynamically regulated phenotypes? Cancer research. 2012; 72: 576-80.
NPL 2: Sharma S V, Lee D Y, Li B, Quinlan M P, Takahashi F, Maheswaran S, et al. A chromatin-mediated reversible drug-tolerant state in cancer cell subpopulations. Cell. 2010; 141:69-80.
NPL 3: Rappa G, Fodstad O, Lorico A. The stem cell-associated antigen CD133 (Prominin-1) is a molecular therapeutic target for metastatic melanoma. Stem Cells. 2008; 26: 3008-17.

### SUMMARY OF INVENTION

### Technical Problem

It is an object of the present invention to provide a novel method for determining cancer tissue malignancy, a novel cancer prognosis evaluation method, novel cancer antigenic peptides, a novel method for producing a cell composition for adoptive immunity, and a novel cancer preventing, cancer treating, cancer metastasis suppressing, or cancer recurrence suppressing agent.

### Solution to Problem

The present inventors previously reported that effector T cells primed with tumor antigens in tumor-draining lymph nodes possessed potent antitumor therapeutic efficacy in brain, pulmonary, and skin metastasis models (Kagamu H, Shu S. Purification of L-selectin (low) cells promotes the generation of highly potent CD4 antitumor effector T lymphocytes. J Immunol. 1998; 160:3444-52., Fujita N, Kagamu H, Yoshizawa H, Itoh K, Kuriyama H, Matsumoto N, et al. CD40 ligand promotes priming of fully potent antitumor CD4(+) T cells in draining lymph nodes in the presence of apoptotic tumor cells. J Immunol. 2001; 167:5678-88.). More recently, the present inventors focused on one of the CSC markers, CD133, and successfully purified CD133-positive melanoma cells, which account for less than 1% of the total melanoma cells. The CD133-positive melanoma cells had the CSC characteristics. The present inventors found that vaccination with the melanoma CSC induced specific CD8-positive T cells, including type 17 T helper (Th17) cells and Th1 cells. In particular, melanoma CSC-specific CD4-positive T cells drove long-lasting accumulation of effector T cells and active dendritic cells with highly expressed MHC class II in tumor tissues, and exhibited a strong anti-tumor effect. Regulatory T cells (Treg), typically seen in tumor tissues, were not induced in mice injected with melanoma CSC-specific CD4-positive T cells. Moreover, this treatment eradicated CD133-positive tumor cells, thereby curing parental melanomas. These results suggest that CD133-positive melanoma cells possess specific immunogenic antigens and that the antigen-specific T cells have an unprecedented level of anti-tumor activity capable of eradicating CSC.

To elucidate the immunogenic proteins that are preferentially expressed in CD133-positive tumor cells, the present inventors compared protein expression using two-dimensional electrophoresis analyses, thereby identifying four proteins. A Mascot search based on mass spectrometry (MS/MS) analysis data identified one of those proteins as DEAD/H (Asp-Glu-Ala-Asp/His) box polypeptide 3, X-linked (DDX3X).

This protein is a member of the DEAD-box family of ATP-dependent RNA helicases (DEAD box helicases) and is located on the X chromosome. DEAD-box helicases have multiple functions, including RNA splicing, mRNA export from nucleus to cytoplasm, transcriptional and translational regulation, RNA decay, and ribosome biogenesis (Rocak S, Linder P. DEAD-box proteins: the driving forces behind RNA metabolism. Nature reviews Molecular cell biology. 2004; 5: 232-41). DDX3X is evolutionarily well conserved from yeast to humans, suggesting that it is essential for cell survival. It has a homologue, DDX3Y, on the Y chromosome, and both of these genes play a role in embryogenesis. In humans, DDX3X deletion or dysfunction results in impairment of germ cell formations (Matzuk M M, Lamb D J. Genetic dissection of mammalian fertility pathways. Nat Cell Biol. 2002; 4 Suppl: s41-9).

In the present invention, the present inventors found that DDX3X is a major immunogenic target protein of CD133-positive melanoma cells. Vaccination with synthesized DDX3X exhibited tumor regression in a skin melanoma treatment model. DDX3X is strongly expressed in human cancer cell lines that express CSC markers, but faintly expressed in normal human epithelial cells and normal human endothelial cells.

From these results, the present inventors envisaged that:

DDX3X expression levels in cancer tissues may be used as an index of cancer malignancy;

the presence or absence of DDX3X-specific T cells in the blood of a cancer patient may be used as an index cancer prognosis evaluation;

a partial peptide (fragment) of DDX3X may be used as a cancer vaccine; and

anti-DDX3X immunotherapy may be a promising strategy in the efforts to eradicate CSC, thereby curing cancer.

The present invention was completed after further studies.

The present invention includes the following aspects.

### Item 1.

A cancer malignancy evaluation method, comprising:

the step of measuring a DDX3X expression level in a cancer tissue; and

the step of evaluating malignancy of the cancer tissue by using the DDX3X expression level.

### Item 2.

A cancer malignancy evaluation kit, comprising:

an antibody against DDX3X, or a polynucleotide that specifically binds to DDX3X mRNA or corresponding cDNA,

wherein the antibody or the polynucleotide is used for measurement of a DDX3X expression level in a cancer tissue.

### Item 3.

A cancer prognosis evaluation method, comprising:

the step of detecting DDX3X-specific T cells in the blood of a cancer patient; and

the step of evaluating cancer prognosis by using the detection result.

### Item 4.

A cancer prognosis evaluation kit, comprising:

DDX3X or a partial peptide thereof,

wherein the DDX3X or a partial peptide thereof is used for detection of DDX3X-specific T cells in the blood of a cancer patient.

### Item 5.

A peptide consisting of:

a sequence of 9 to 20 contiguous amino acids that includes the amino acid sequence represented by any of SEQ ID NOS: 2 to 87 in the amino acid sequence of SEQ ID NO: 1, or

an amino acid sequence essentially the same as such an amino acid sequence.

### Item 6.

A peptide according to Item 5, wherein the peptide is a cancer antigenic peptide.

### Item 7.

A cancer vaccine comprising the peptide of Item 5.

### Item 7-2.

A peptide according to Item 5 which is for use in vaccination against cancer.

### Item 8.

A cancer vaccine according to Item 7, wherein the vaccine is used for preventing or treating cancer, or for suppressing cancer metastasis or cancer recurrence.

### Item 9.

An adoptive immunity cell producing method comprising the step of pulsing a cell having an antigen-presenting ability with DDX3X or a partial peptide thereof.

### Item 10.

An antigen-presenting cell pulsed with DDX3X or a partial peptide thereof.

### Item 11.

A DDX3X-specific T cell inducer comprising the antigen-presenting cell of Item 10 as an active component.

### Item 11-2.

An antigen-presenting cell according to Item 10, wherein the antigen-presenting cell is used to induce DDX3X-specific T cells.

### Item 12.

A method of producing an adoptive immunity cell composition comprising:

the step of exposing a cell having an antigen-presenting ability to DDX3X or a partial peptide thereof to obtain a cell presenting an antigen derived from the DDX3X or a partial peptide thereof; and

the step of inducing a DDX3X-specific T cell with the antigen-presenting cell.

### Item 13.

A method according to Item 12, wherein the DDX3X-specific T cell is a DDX3X specific CD4-positive T cell.

### Item 14.

A cancer preventing, cancer treating, cancer metastasis suppressing, or cancer recurrence suppressing agent comprising a compound that inhibits DDX3X expression or activity.

### Item 14-2.

A compound that inhibits DDX3X expression or activity for use in preventing or treating cancer, or for suppressing cancer metastasis or cancer recurrence.

### Advantageous Effects of Invention

The peptide of the present invention is a cancer antigen, and can be used to provide a method for determining cancer malignancy, a cancer prognosis evaluation method, cancer antigenic peptides, a method for producing a cell composition for adoptive immunity, and a cancer preventing, cancer treating, cancer metastasis suppressing, or cancer recurrence suppressing agent, among others.

### BRIEF DESCRIPTION OF DRAWINGS

FIG. 1 represents the amino acid sequence of human DDX3X.

FIG. 2 is a graph representing IFN-γ production by CD8-positive T cells (Example 1).

FIG. 3 represents graphs depicting IFN-γ and IL-17 production by CD4-positive T cells (Example 1).

FIG. 4 is a graph representing IFN-γ production by CD8-positive T cells (Example 1).

FIG. 5 represent graphs depicting IFN-γ and IL-17 production by CD4-positive T cells (Example 1).

FIG. 6 represents tumor growth curves of vaccinated mice (Example 2).

FIG. 7 represents a tumor growth curve of vaccinated mice with established skin tumors (Example 3).

FIG. 8 represents a tumor growth curve of vaccinated mice (Example 4).

FIG. 9 is a graph representing IFN-γ production by stimulation with DD3C partial peptides (Example 5).

FIG. 10 represents graphs depicting IFN-γ production by stimulation with DD3C partial peptides (Example 6).

FIG. 11 is a graph (B) representing a tumor growth curve of vaccinated mice (Example 7).

FIG. 12 represents flow cytometry graphs of CD133 expression (Example 8).

FIG. 13 represents immunoblots from tumor cells using antibodies against DDX3X and β-actin (Example 8).

FIG. 14 shows photographs taken in the course of injury repair in cell injury repair experiment (Example 9).

FIG. 15 shows photographs of spheroids taken to examine spheroid formation (Example 11).

### DESCRIPTION OF EMBODIMENTS

As used herein, “cancer” refers to abnormal and uncontrolled proliferation of cells in an organism. Examples include solid tumors (for example, carcinoma, and sarcoma), lymphoma, and leukemia.

More specific examples include:

childhood brain tumors such as astroglioma, malignant medulloblastoma, germ cell tumor, craniopharyngioma, and ependymoma;

adult brain tumors such as glioma, neuroglioma, meningioma, pituitary adenoma, neurilemoma;
head and neck cancers such as maxillary sinus cancer, pharyngeal cancer (nasopharyngeal carcinoma, mesopharyngeal carcinoma, hypopharyngeal carcinoma), laryngeal cancer, oral cancer, lip cancer, tongue cancer, and parotid cancer;
thoracic cancers and tumors such as small cell lung cancer, non-small cell lung cancer, thymoma, and mesothelioma;
gastrointestinal cancers and tumors such as esophageal cancer, liver cancer, primary hepatic cancer, gallbladder cancer, bile duct cancer, stomach cancer, large bowel cancer, colonic cancer, rectal cancer, anal cancer, pancreatic cancer, and pancreatic endocrine tumor;
urinary organ cancers and tumors such as penile cancer, renal pelvis and ureteral cancer, renal cell cancer, testicular tumor, prostatic cancer, bladder cancer, Wilms tumor, and urothelial cancer;
gynecologic cancers and tumors such as vulvar cancer, uterine cervical cancer, corpus uteri cancer, endometrial cancer, uterine sarcoma, chorionic cancer, vaginal cancer, breast cancer, ovarian cancer, and ovarian germ cell tumor;
adult and childhood soft tissue sarcoma;
bone tumors such as osteosarcoma and Ewing's tumor;
endocrine tissue cancers and tumors such as adrenocortical cancer and thyroid cancer;
malignant lymphoma and leukemia such as malignant lymphoma, non-Hodgkin's lymphoma, Hodgkin's disease, multiple myeloma, plasmacytic tumor, acute myelogenous leukemia, acute lymphatic leukemia, adult T cell leukemia lymphoma, chronic myelogenous leukemia, and chronic lymphatic leukemia;
skin cancers and tumors such as chronic myeloproliferative disorders, malignant melanoma (melanoma), squamous cell cancer, basal cell cancer, and mycosis fungoides;
and metastatic foci of these tumors and cancers.

The present invention is particularly suited for application in, for example, thoracic cancers and tumors such as small cell lung cancer, and non-small cell lung cancer; skin cancers and tumors such as malignant melanoma (melanoma); and gynecologic cancers and tumors such as breast cancer.

DDX3X is DEAD/H (Asp-Glu-Ala-Asp/His) box polypeptide 3, X-linked.

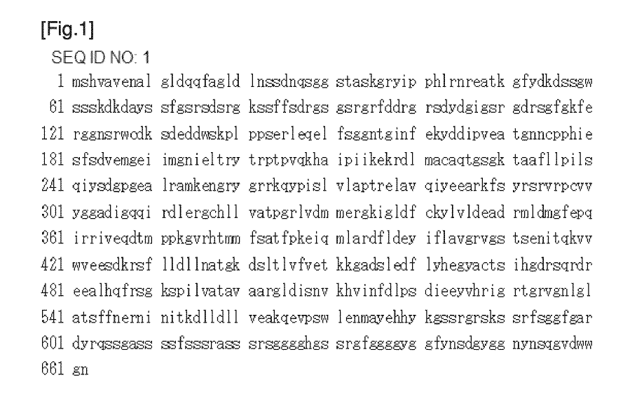

As noted above, this protein is a member of the DEAD-box family of ATP-dependent RNA helicases (DEAD box helicases) and is located on the X chromosome. The amino acid sequence is known. The sequence of human DDX3X (UniProtKB/Swiss-Prot: 000571.3) is represented in FIG. 1 (SEQ ID NO: 1).

All abbreviations, including base sequences (nucleotide sequences), and nucleic acids and amino acids used herein follow the rules specified by IUPAC-IUB [IUPAC-IUB communication on Biological Nomenclature, Eur. J. Biochem., 138; 9 (1984)], Guidelines for the Preparation of Specification which Contains Nucleotide and/or Amino Acid Sequence (JPO), and the conventional notation used in the art.

The base sequences as they occur in this specification are reported from the 5′ to the 3′ end, unless otherwise stated.

The amino acid sequences as they occur in this specification are reported from the N terminal to the C terminal, unless otherwise stated.

As used herein, “gene” is inclusive of double-stranded DNA and single-stranded DNA (sense strand), and single-stranded DNA (antisense strand) having a complementary sequence to the sense strand, and fragments thereof, unless otherwise stated. Further, the use of the term “gene” herein does not distinguish between regulatory region, coding region, exon, and intron, unless otherwise stated.

As used herein, “nucleotide” (or “polynucleotide”) has the same meaning as nucleic acid, and includes both DNA and RNA. These may be double-stranded or single-stranded. By “nucleotide (or “polynucleotide”) of a certain sequence, it inclusively means a nucleotide (or polynucleotide) having a complementary sequence, unless otherwise stated.

As used herein, “polynucleotide” is inclusive of oligonucleotide, unless otherwise stated.

Further, “nucleotide” (or “polynucleotide”) is inclusive of modified nucleic acid or nucleic acid analog (for example, PNA, and LNA), unless otherwise stated.

When the “nucleotide” (or “polynucleotide”) is an RNA, the letter “T” in the bases of the Sequence Listing should be read as “U”.

As used herein, “cDNA” encompasses both single-stranded DNA (single-stranded cDNA) having a base sequence complementary to mRNA, and double-stranded DNA (double-stranded cDNA) of the single-stranded cDNA and its complementary strand, unless otherwise stated.

As used herein, “specific hybridization” means that hybridization occurs without significant cross hybridization with other polynucleotides in a sample under ordinary hybridization conditions, preferably under stringent hybridization conditions (for example, under the conditions described in Sambrook, et al., Molecular Cloning, Cold Spring Harbour Laboratory Press, New York, USA, 2nd Ed., 1989). In a specific example of such stringent hybridization conditions, a positive hybridization signal is observed after heating in a 6×SSC, 0.5% SDS, and 50% formamide solution at 42° C., followed by washing in a 0.1×SSC, 0.5% SDS solution at 68° C.

As used herein, “protein” encompasses both modified proteins (for example, with sugar chains) and unmodified proteins, unless otherwise stated. This applies also to proteins not particularly specified as proteins.

As used herein, peptides derived from DDX3X or partial peptides thereof are also referred to as DDX3X-derived peptides.

As used herein, partial peptides (or fragments) of DDX3X means peptides containing a partial amino acid sequence of DDX3X.

### Cancer Malignancy Evaluation Method

The cancer malignancy evaluation method of the present invention includes the step of measuring a DDX3X expression level in a cancer tissue, and the step of evaluating cancer malignancy by using the DDX3X expression level.

As used herein, “cancer malignancy” is the indication that how soon the cancer, clinically, kills the host. Specifically, it can be regarded as the extent of mortality due to cancer, the likelihood of metastasis, the extent of cancer prognosis, or the difficulty of cancer treatment.

As used herein, “cancer tissue” may be, for example, a cancer tissue collected from a patient for testing purposes, a cancer tissue removed by surgery, or a part of such cancer tissues.

The DDX3X expression level measurement may be performed by using any means capable of distinguishing between the DDX3X expression level in a high malignancy cancer tissue and the DDX3X expression level in a low malignancy cancer tissue.

In an embodiment of the present invention, the DDX3X expression level measurement may be performed by measuring the amount of the protein DDX3X. Specifically, for example, the protein is extracted or prepared from cancer tissue using an ordinary method, as required, and the DDX3X expression level is measured by using methods exemplified below. The protein may be extracted or prepared by using, for example, a commercially available kit.

The method used to measure DDX3X amount is not particularly limited, as long as the protein amount can be specifically measured. Examples include western blotting, ELISA, fluorescence antibody method, and protein array (protein chip) method.

In ELISA, for example, a solution containing the protein extracted or prepared from a cancer tissue is adsorbed to the solid surface of microplate wells, and the DDX3X amount is measured through enzyme reaction after applying antibodies against DDX3X.

In the protein array method, for example, a protein array (for example, an antibody array (antibody chip)) having antibodies against DDX3X is prepared, and proteins extracted from a cancer tissue are applied to the protein array. After antibody-antigen reaction, the amount of the DDX3X that has bound to the antibodies is measured by using a method such as ELISA.

The antibodies may be, for example, polyclonal antibodies, or monoclonal antibodies.

The antibodies may be, for example, antibody fragments, such as Fab fragment and F(ab′)2 fragment, that can specifically bind to antigens.

The antibodies can be produced by using known methods.

For example, when the antibodies are polyclonal antibodies, the antibodies can be prepared from the serum of an immunized animal by using a common technique, after immunizing a non-human animal such as rabbit with the DDX3X or a partial peptide thereof prepared through expression in Escherichia coli or the like using a known method, or with DDX3X or a partial peptide thereof synthesized by using a known method.

On the other hand, when the antibodies are monoclonal antibodies, the DDX3X or a partial peptide thereof prepared through expression in Escherichia coli or the like using a known method is used as antigen, and the antibodies may be prepared from a hybridoma prepared by fusing myeloma with antibody-producing cells obtained through immunization of a non-human mammal such as mouse using the antigen prepared as above.

Preferred for use as the DDX3X partial peptide in the present invention is, for example, the peptide of the present invention described below.

The DDX3X or partial peptides thereof may be produced by using a common chemical synthesis technique according to the available amino acid sequence information. Such techniques include ordinary liquid-phase or solid-phase peptide synthesis techniques. Examples of such peptide synthesis techniques include techniques described in Peptide Synthesis (Maruzen, 1975), and Peptide Synthesis, Interscience, New York, (1996). A known chemical synthesis device, for example, such as a peptide synthesizer (Applied Biosystems) also may be used to produce the DDX3X or partial peptides thereof.

The DDX3X, or a peptide consisting of a partial amino acid sequence of DDX3X used as antigen also may be produced with the use of an expression vector, a cloning vector, and the like, using a common genetic engineering technique that includes procedures such as DNA cloning based on the base sequence information of the coding gene, plasmid construction, transfection into a host, transfectant culture, and collection of the protein from the cultured product.

The recombinant vector may be obtained by incorporating the DDX3X or a polynucleotide encoding a partial peptide thereof into a suitable vector DNA.

The vector DNA may be appropriately selected according to the type of host, and intended use. The vector DNA may be DNA found in nature, or natural DNA with partial deletion of DNA portions other than regions needed for proliferation. Examples of the vector DNA include vectors derived from chromosomes, episomes, or viruses. Specific examples include bacteria plasmids, bacteriophages, transposons, yeast episomes, insertion elements, yeast chromosome elements, vectors derived from viruses (for example, such as baculovirus, papovavirus, SV40, vaccinia virus, adenovirus, fowlpox virus, pseudorabies virus, and retrovirus) and vectors with combinations of these viruses, and vectors derived from genetic elements of plasmids and bacteriophages (for example, such as cosmids and phagemids).

A recombinant vector containing the polynucleotide can be obtained upon insertion of the polynucleotide into vector DNA by using a known method. Specifically, for example, DNA and vector DNA are cut at specific sites with suitable restriction enzymes, and mixed and religated with ligase. Alternatively, a suitable linker may be ligated to the polynucleotide, and inserted into the multiple cloning site of vector DNA suited for intended use to obtain the recombinant vector.

A transfectant with the recombinant vector may be obtained by introducing the polynucleotide-containing recombinant vector into a known host, for example, bacteria such as Escherichia coli (for example, K12) and Bacillus bacteria (for example, MI114), yeasts (for example, AH22), insect cells (for example, Sf cells), and animal cells (for example, COS-7 cells, Vero cells, and CHO cells), using a known method.

Considering gene stability, chromosomal integration can preferably be used as the gene introduction method. For convenience, an autonomous replication system using extranuclear genes may be used. Introduction of the vector DNA into host cells may be performed according to standard methods, for example, such as the method described in Molecular Cloning: A Laboratory Manual (Sambrook et al., Cold Spring Harbour Laboratory Press, Cold Spring Harbour, N.Y., 1989). Specific examples include calcium phosphate transfection, DEAE-dextran-mediated transfection, microinjection, cationic lipid-mediated transfection, electroporation, transduction, scrape loading, and ballistic introduction.

DDX3X is also commercially available.

The anti-DDX3X antibodies are also commercially available.

The antibodies used for the measurement of DDX3X amount may be labeled by using known labeling methods, for example, such as enzyme labeling, radiolabeling, and fluorescent labeling, or may be modified with biotin and the like.

In another embodiment of the present invention, the DDX3X expression level measurement may be performed by measuring the mRNA amount of DDX3X. Specifically, for example, mRNA is extracted or prepared from the cancer tissue by using an ordinary method, as required, and the DDX3X mRNA amount is measured, for example, by using the method exemplified below. The extraction or preparation of mRNA may be performed by using, for example, a commercially available kit.

The method used for the DDX3X mRNA amount measurement is not particularly limited, as long as it can measure the level of specific mRNA. For example, known methods using polynucleotide probes or primers that specifically bind to the DDX3X mRNA or corresponding cDNA may be used, including, for example, southern blotting, in situ hybridization, comparative genomic hybridization (CGH), quantitative PCR (e.g., real-time PCR), and the Invader (the product from HOLOGIC, USA) method.

In the micro array method, for example, a nucleic acid array (nucleic acid chip) with a probe that specifically binds to DDX3X mRNA is prepared, and an mRNA sample extracted from cancer tissue and labeled with fluorescent or other labels is applied to the nucleic acid array. Signals from the labeled DDX3X mRNA that has bound to the probe are then measured and analyzed.

In real-time PCR, for example, the mRNA extracted or prepared from cancer tissue is reverse-transcribed into cDNA using reverse transcriptase. By using the cDNA as a template, predetermined regions are PCR amplified with primers that specifically bind to DDX3X cDNA, and amplification products are monitored in real time as they are produced.

The probes used for the measurement of mRNA amount are designed to allow for specific hybridization with DDX3X mRNA or cDNA.

The polynucleotide as the probe is preferably a polynucleotide selected from the group consisting of the whole base sequence or a partial base sequence of DDX3X mRNA, or a complementary sequence thereof, and such polynucleotides formed by the deletion, substitution, or addition of one to several (for example, 1 to 10, 1 to 5, 1 to 3) bases of the polynucleotides. The probe polynucleotides are typically 15 to 500 bases long, preferably 20 to 200 bases long, more preferably 20 to 50 bases long.

The probe polynucleotides may include labels, for example, such as a fluorescent dye, an enzyme, a protein, a radioisotope, and a chemiluminescence substance, appropriately added to enable DDX3X mRNA amount measurement.

The primers used for mRNA amount measurement by quantitative PCR or the like are designed to allow for specific hybridization with DDX3X mRNA or cDNA. The methods used to design the primers are not particularly limited, and, for example, known methods may be used with algorithm or software intended for primer design applications. The primers are typically used as forward and reverse primer sets.

The primers are preferably polynucleotides selected from, for example, polynucleotides consisting of the whole sequence or a partial sequence of DDX3X mRNA, or a complementary sequence thereof, and such polynucleotides formed by the deletion, substitution, or addition of one to several (for example, 1 to 10, 1 to 5, 1 to 3) bases of the polynucleotides. The primer polynucleotides are typically 15 to 30 bases long.

The primer polynucleotides may include labels, for example, such as a fluorescent dye, an enzyme, a protein, a radioisotope, and a chemiluminescence substance, appropriately added to enable DDX3X mRNA amount measurement.

The polynucleotides can be produced, for example, by using genetic engineering techniques [for example, Methods in Enzymology, 2005; 392: 24-35, 73-96,173-185, 405-419.; Nucleic Acids Res. 1984; 12:9441; New Biochemical Experiment Course 1, Gene Research Technique II, The Japanese Biochemical Society, p. 105 (1986)], chemical synthesis means such as the phosphotriester method and the phosphoamidide method [J Am Chem Soc. 1967; 89(2): 450-3.; J Am Chem Soc. 1967; 89 (26): 7146-7147.], and combinations of these methods. RNA synthesis may also be performed according to the phosphoramidide method, using, for example, a commercially available high throughput DNA synthesizer AB13900 (Applied Biosystems) with RNA synthesis reagents.

The polynucleotides may also be obtained through consignment to companies or sections of companies in service of synthesizing polynucleotides.

The DDX3X expression level measurement also may be performed by counting DDX3X-expressing cells in cancer tissue. Counting of DDX3X-expressing cells may be performed by observing DDX3X-expressing cells in an immunohistostained cancer tissue, or by counting DDX3X-expressing cells in cancer tissue by using a technique such as flow cytometry, using anti-DDX3X antibodies labeled with, for example, a fluorescent dye, an enzyme, a protein, a radioisotope, or a chemiluminescence substance.

In the cancer malignancy evaluation method of the present invention, cancer tissue is evaluated as having high malignancy when the measured DDX3X expression levels are high, and low malignancy when the measured DDX3X expression levels are low.

The DDX3X expression level and the level of cancer tissue malignancy can be correlated to each other, for example, by using statistical methods (for example, Student's t-test, and Kaplan-Meier method), based on the DDX3X expression levels in, for example, a non-cancer tissue, a high-malignancy cancer tissue identified by conventional evaluation, and a low-malignancy cancer tissue identified by conventional evaluation.

The cancer malignancy evaluation method of the present invention may be performed with other cancer malignancy evaluation methods.

### Cancer Malignancy Evaluation Kit

The cancer malignancy evaluation kit of the present invention can be used for the cancer malignancy evaluation method of the present invention.

The cancer malignancy evaluation kit of the present invention comprises antibodies against DDX3X, or polynucleotides that specifically bind to DDX3X mRNA or corresponding cDNA.

In an embodiment of the present invention, the cancer malignancy evaluation kit includes antibodies against DDX3X.

The antibodies are used to measure the amount of the protein DDX3X.

The same antibodies described in conjunction with the cancer malignancy evaluation method may be used as the antibodies of the cancer malignancy evaluation kit.

The antibodies may form a protein array (for example, an antibody array, and an antibody chip). The protein array has a substrate and the antibodies, and the antibodies are disposed on the substrate. The substrate is not particularly limited, as long as protein can be disposed thereon. Examples include a glass plate, a nylon membrane, microbeads, a silicon chip, and a capillary. The protein array (antibody chip) can be produced by immobilizing the antibodies on the substrate by using a method commonly used for protein array production, for example, such as a method using the inkjet technique.

In another embodiment of the present invention, the cancer malignancy evaluation kit of the present invention includes polynucleotides that specifically bind to DDX3X mRNA or corresponding cDNA.

The polynucleotides are used to measure DDX3X mRNA amount.

The same polynucleotides described in conjunction with the cancer malignancy evaluation method may be used as the polynucleotides of the cancer malignancy evaluation kit.

The polynucleotides may form a nucleic acid array. The nucleic acid array has a substrate and the polynucleotides, and the polynucleotides are disposed on the substrate. The polynucleotides may be the same polynucleotides described in conjunction with the cancer malignancy evaluation method. The substrate is not particularly limited, as long as nucleic acid can be disposed thereon. Examples include a glass plate, a nylon membrane, microbeads, a silicon chip, and a capillary. The nucleic acid array can be produced by immobilizing the polynucleotides on a substrate by using a method commonly used for nucleic acid array production, for example, such as a method using a commercially available spotter, and a method using the inkjet technique.

The cancer malignancy evaluation kit of the present invention may include an enzyme, a buffer, a reagent, or a manual as may be selected according to intended use or form.

### Cancer Prognosis Evaluation Method

The cancer prognosis evaluation method of the present invention includes:

the step of detecting DDX3X-specific T cells in the blood of a cancer patient; and

the step of evaluating cancer prognosis by using the detection result.

As used herein, “cancer prognosis” means the probable course of cancer.

Preferably, the detection of DDX3X-specific T cells in the blood of a cancer patient is performed by detecting DDX3X-specific T cells in a blood sample obtained from a cancer patient.

The blood sample may be obtained by using the common method.

Detection of DDX3X-specific T cells in a blood sample can be performed by using common antigen specific T cell detection methods, including, for example, antigen-dependent proliferation analysis (such as ³H-thymidine incorporation assay), cytotoxic measurement (such as ⁵¹Cr release assay), MHC-peptide-tetramer staining, enzyme-linked immunospot (ELISPOT) assay, and intracellular cytokine assay.

The antigens used in these detection methods may be the same antigens described above in conjunction with the cancer malignancy evaluation method.

Prognosis is evaluated as being good when DDX3X-specific T cells are detected in the blood of a cancer patient, and bad when DX3X-specific T cells are not detected.

The cancer prognosis evaluation method of the present invention may be performed with other cancer prognosis evaluation methods.

### Cancer Prognosis Evaluation Kit

The cancer prognosis evaluation kit of the present invention may be used for the cancer malignancy evaluation method of the present invention.

The cancer prognosis evaluation kit of the present invention comprises DDX3X or a partial peptide thereof.

The DDX3X or a partial peptide thereof is used for the detection of DDX3X-specific T cells in the blood of a cancer patient.

The DDX3X or partial peptides thereof are the same DDX3X or partial peptides thereof described in conjunction with the cancer malignancy evaluation method. The peptide of the present invention (described below) is preferably used as the DDX3X or a partial peptide thereof.

### Peptide

The peptide of the present invention consists of a sequence of 9 to 20 contiguous amino acids that comprises the amino acid sequence represented by any of the following SEQ ID NOS: 2 to 87 in the amino acid sequence of SEQ ID NO: 1, or an amino acid sequence essentially the same as such an amino acid sequence.

SEQ ID NO: 1 is the amino acid sequence of DDX3X, as noted above.

SEQ ID NO: 2:
FLLDLLNAT
SEQ ID NO: 3:
NITQKVVWV
SEQ ID NO: 4:
IQMLARDFL
SEQ ID NO: 5:
TFPKEIQML
SEQ ID NO: 6:
KYDDIPVEA
SEQ ID NO: 7:
RYIPPHLRN
SEQ ID NO: 8:
RNINITKDL
SEQ ID NO: 9:
KQYPISLVL
SEQ ID NO: 10:
IGLDFCKYL
SEQ ID NO: 11:
IELTRYTRP
SEQ ID NO: 12:
TRYTRPTPV
SEQ ID NO: 13:
MGNIELTRY
SEQ ID NO: 14:
LVLAPTREL
SEQ ID NO: 15:
YPISLVLAP
SEQ ID NO: 16:
QYPISLVLA
SEQ ID NO: 17:
LEDFLYHEGY
SEQ ID NO: 18:
FLDEYIFLA
SEQ ID NO: 19:
LLVEAKQEV
SEQ ID NO: 20:
FLLPILSQI
SEQ ID NO: 21:
DFLDEYIFL
SEQ ID NO: 22:
SHVAVENAL
SEQ ID NO: 23:
VAVENALGL
SEQ ID NO: 24:
ALGLDQQFA
SEQ ID NO: 25:
LGLDQQFAG
SEQ ID NO: 26:
GLDQQFAGL
SEQ ID NO: 27:
DQQFAGLDL
SEQ ID NO: 28:
NSSDNQSGG
SEQ ID NO: 29:
KGRYIPPHL
SEQ ID NO: 30:
PHLRNREAT
SEQ ID NO: 31:
RGRGDYDGI
SEQ ID NO: 32:
YDGIGSRGD
SEQ ID NO: 33:
RSGFGKFER
SEQ ID NO: 34:
KPLPPSERL
SEQ ID NO: 35:
LFSGGNTGI
SEQ ID NO: 36:
FSGGNTGIN
SEQ ID NO: 37:
INFEKYDDI
SEQ ID NO: 38:
YDDIPVEAT
SEQ ID NO: 39:
TGNNCPPHI
SEQ ID NO: 40:
EIIMGNIEL
SEQ ID NO: 41:
IIMGNIELT
SEQ ID NO: 42:
IPIIKEKRD
SEQ ID NO: 43:
GSGKTAAFL
SEQ ID NO: 44:
TAAFLLPIL
SEQ ID NO: 45:
AAFLLPILS
SEQ ID NO: 46:
IYADGPGEA
SEQ ID NO: 47:
LAVQIYEEA
SEQ ID NO: 48:
IYEEARKFS
SEQ ID NO: 49:
RPCVVYGGA
SEQ ID NO: 50:
CVVYGGADI
SEQ ID NO: 51:
LLVATPGRL
SEQ ID NO: 52:
ATPGRLVDM
SEQ ID NO: 53:
GLDFCKYLV
SEQ ID NO: 54:
LDFCKYLVL
SEQ ID NO: 55:
LVLDEADRM
SEQ ID NO: 56:
VLDEADRML
SEQ ID NO: 57:
GFEPQIRRI
SEQ ID NO: 58:
FSATFPKEI
SEQ ID NO: 59:
YIFLAVGRV
SEQ ID NO: 60:
RVGSTSENI
SEQ ID NO: 61:
ATGKDSLTL
SEQ ID NO: 62:
SLTLVFVET
SEQ ID NO: 63:
FLYHEGYAC
SEQ ID NO: 64:
LYHEGYACT
SEQ ID NO: 65:
LHQFRSGKS
SEQ ID NO: 66:
QFRSGKSPI
SEQ ID NO: 67:
ILVATAVAA
SEQ ID NO: 68:
TAVAARGLD
SEQ ID NO: 69:
ISNVKHVIN
SEQ ID NO: 70:
LPSDIEEYV
SEQ ID NO: 71:
EYVHRIGRT
SEQ ID NO: 72:
LGLATSFFN
SEQ ID NO: 73:
TSFFNERNI
SEQ ID NO: 74:
FFNERNINI
SEQ ID NO: 75:
NITKDLLDL
SEQ ID NO: 76:
DLLDLLVEA
SEQ ID NO: 77:
EVPSWLENM
SEQ ID NO: 78:
AYEHHYKGS
SEQ ID NO: 79:
EHHYKGSSR
SEQ ID NO: 80:
SRFSGGFGA
SEQ ID NO: 81:
FGARDYRQS
SEQ ID NO: 82:
GGGYGGFYN
SEQ ID NO: 83:
GGYGGFYNS
SEQ ID NO: 84:
GGFYNSDGY
SEQ ID NO: 85:
SDGYGGNYN
SEQ ID NO: 86:
GGNYNSQGV
SEQ ID NO: 87:
NYNSQGVDW

For example, the amino acid sequences represented by SEQ ID NOS: 88 to 92 below also may be used, in addition to the amino acid sequences of SEQ ID NOS: 2 to 87.

SEQ ID NO: 88:
KQYPISLVLAPTREL
SEQ ID NO: 89:
EIIMGNIELTRYTRPTPV
SEQ ID NO: 90:
KGADSLEDFLYHEGY
SEQ ID NO: 91:
FVETKKGADSLEDFLYHEGY

The terminal glutamine residue in these sequences may be cyclized to form a pyroglutamic acid. An example of such a sequence is SEQ ID NO: 92: pyroEYPISLVLA.

As used herein, “the peptide consisting of an amino acid sequence essentially the same as the sequence of 9 to 20 contiguous amino acids that comprises the amino acid sequence represented by any one of SEQ ID NOS: 2 to 87” may be the peptide consisting of “a sequence of 9 to 20 contiguous amino acids that comprises the amino acid sequence represented by any one of SEQ ID NOS: 2 to 87” which has the substitution, deletion, and/or addition of one to several (e.g., 1 to 10, 1 to 5, 1 to 3, 1 to 2, and 1) amino acid residues.

As used herein, the “amino acid sequence essentially the same as” may be an amino acid sequence which has amino acid sequence identity of 80% or more (preferably 85% or more, more preferably 88% or more).

The substitution may be conservative substitution.

Examples of conservative substitutions include a substitution between aspartic acid and glutamic acid, a substitution between arginine, lysine, and histidine, a substitution between triptophan and phenylalanine, a substitution between phenylalanine and valine, a substitution between leucine, isoleucine, and alanine, and a substitution between glycine and alanine.

For example, preferred among the amino acid sequences represented by any one of SEQ ID NOS: 2 to 87 is the amino acid sequence represented by any one of SEQ ID NOS: 2 to 17, and 40 and 41, more preferably the amino acid sequence represented by any one of SEQ ID NOS: 9, 11 to 17, 40, and 41.

The cancer antigenic peptide of the present invention consists of preferably 9 to 15 amino acid residues, more preferably 9 to 12 amino acid residues, further preferably 9 to 11 amino acid residues, particularly preferably 10 amino acid residues.

Preferably, the cancer antigenic peptide of the present invention consists of a sequence of 9 to 20 contiguous amino acids that comprises the amino acid sequence represented by any one of SEQ ID NOS: 2 to 17, and 88 to 92 in the amino acid sequence of SEQ ID NO: 1.

Particularly preferably, the cancer antigenic peptide of the present invention consists of the amino acid sequence represented by SEQ ID NO: 17, 88, or 89.

The cancer antigenic peptide of the present invention can be prepared as a peptide isolated by using a known method, as described above for the DDX3X or the peptide consisting of a partial amino acid sequence of DDX3X in conjunction with the cancer tissue malignancy evaluation method.

As used herein, “isolated” means a non-naturally occurring state.

The peptide of the present invention may be in the form of a salt. Examples of such salts include salts of inorganic acids such as hydrochloric acid and phosphoric acid, and salts of organic acids such as acetic acid and tartaric acid.

The peptide of the present invention may be used in the form of a conjugate by being added with sugar, polyethylene glycol, lipid, or the like, or in the form of a derivative such as by radioisotopes, or a polymer.

The peptide of the present invention may be a cancer antigenic peptide.

As used herein, “cancer antigenic peptide” may mean a peptide that can be recognized by cancer-specific cytotoxic T cells (CTL), and that can induce and/or activate CTL.

As used herein, “recognized” may mean perceived as being different by a recognizing substance, and, for example, the recognizing substance binding to the target perceived as being different. As used herein, “recognizing the peptide” means binding CTL to human leukocyte antigen (HLA) and the peptide via T cell receptors.

As used herein, “activate” may mean further enhancing or activating the activity or effect of some substance, or the state of such a substance having some activity or effect. Specifically, “activating CTL” means that CTL recognizes the peptide presented by HLA, and produces an effector, for example, such as IFN-γ, or that CTL exhibits cytotoxicity against the recognized target cells.

As used herein, “induce” may mean producing activity or effect from a substance having essentially no activity or effect, or from the state of such a substance having essentially no activity or effect. Specifically, “inducing antigen specific CTL” may mean causing differentiation and/or proliferation in CTL specifically recognizing some antigen, either in vitro or in vivo.

As used herein, the term “specific” used in conjunction with antibody or antigen refers to the ability to specifically bind to an antibody or an antigen immunologically.

### Cancer Vaccine

The cancer vaccine of the present invention contains the peptide of the present invention as a cancer antigen.

The peptide of the present invention may be prepared into the cancer vaccine either alone or with various carriers.

The dosage form of the cancer vaccine of the present invention may be an orally administered form or a parenterally administered form. Generally, a parenterally administered form is preferred. Examples of the parenterally administered form include a subcutaneous injection, an intramuscular injection, an intravenous injection, and a suppository.

When the cancer vaccine of the present invention is an orally administered form, the peptide of the present invention may be prepared into the cancer vaccine with an excipient that is pharmaceutically acceptable, and that does not interfere with the activity of the peptide of the present invention as cancer antigen. Examples of such excipients include starch, mannitol, lactose, magnesium stearate, cellulose, polymerized amino acid, and albumin.

When the cancer vaccine of the present invention is a parenterally administered form, the peptide of the present invention may be prepared into the cancer vaccine with a carrier that is pharmaceutically acceptable, and that does not interfere with the activity of the peptide of the present invention as cancer antigen. Examples of such carriers include water, common salts, dextrose, ethanol, glycerol, and DMSO.

The cancer vaccine of the present invention may further contain materials such as albumin, a humectant, and/or an emulsifier, as desired.

The peptide of the present invention also may be used with a suitable adjuvant to activate cellular immunity. The cancer vaccine of the present invention may contain such an adjuvant.

The peptide of the present invention may also be used with a compound that enhances the peptide recognition by the cytotoxic T cells (CTL), or with antibodies that immunologically recognize the peptide, for example. The cancer vaccine of the present invention may contain such compounds and/or antibodies.

The cancer vaccine of the present invention may be produced by using the common methods as may be suitable for the dosage form.

Preferably, the cancer vaccine of the present invention is used for preventing or treating cancer, or for suppressing cancer metastasis or cancer recurrence.

The cancer vaccine of the present invention may be administered to humans by using an administration method as may be suitable for the dosage form.

The cancer vaccine of the present invention may be administered to adult humans in a dose of, for example, about 0.01 mg to 100 mg/day, preferably about 0.1 mg to 30 mg/day in terms of the active component peptide of the present invention. The dosing intervals may be appropriately selected according to such factors as the symptom, and the purpose of administration.

### Adoptive Immunity Cell Producing Method

The adoptive immunity cell producing method of the present invention comprises the step of pulsing cells having an antigen-presenting ability with DDX3X or a partial peptide thereof.

Examples of cells having an antigen-presenting ability include dendritic cells, macrophage, and B lymphocytes.

Pulsing may be performed by, for example, incubating cells having an antigen-presenting ability in a medium containing about 1 to 10 μg/ml of DDX3X or a partial peptide thereof at a temperature of about 20 to 30° C. for about 30 minutes to about 1 hour. In this way, cells having the cancer antigenic peptide presented on cell surface for recognition by the DDX3X-specific CTL can be obtained. The cells may be isolated cells.

Preferably, the partial peptide of DDX3X is the peptide of the present invention.

The cells having the DDX3X-derived peptide presented for recognition by the DDX3X-specific CTL may be antigen-presenting cells (APC) presenting the peptide of the present invention.

The APC pulsed with the DDX3X or a partial peptide thereof may be DDX3X-derived peptide-presenting APC, and may be used as a DDX3X-specific T cell inducer.

The APC may be administered as adoptive immunity cells to humans in need of adoptive immunity treatment.

The APC may be cultured by using a known method before being administered to humans.

DDX3X-specific CTL can be induced ex vivo by incubating precursor cells having potential to differentiate into CTL, together with the APC pulsed with the DDX3X or a partial peptide thereof as above. The DDX3X-specific CTL may be isolated cells.

The precursor cells are not particularly limited, as long as they can differentiate into CTL. Examples include peripheral blood mononulclear cells (PBMC), naive cells, and memory cells.

The DDX3X-specific CTL obtained as above may also be administered as adoptive immunity cells to humans in need of adoptive immunity treatment.

The DDX3X-specific CTL may be cultured by using a known method before being administered to humans.

Specifically, in another embodiment of the present invention, the adoptive immunity cell producing method of the present invention comprises:

the step of exposing cells having an antigen-presenting ability to DDX3X or a partial peptide thereof to obtain cells presenting antigens derived from the DDX3X or a partial peptide thereof; and

the step of inducing the DDX3X-specific T cells with the cells.

Preferably, the DDX3X-specific T cells are DDX3X specific CD4-positive T cells.

The adoptive immunity cells obtained as above may be prepared into an adoptive immunity cell composition either directly or with various carriers.

The dosage form of the adoptive immunity cell composition may be an orally administered form or a parenterally administered form. Generally, a parenterally administered form is preferred. Examples of the parenterally administered form include a subcutaneous injection, an intramuscular injection, an intravenous injection, and a suppository.

When the adoptive immunity cell composition is an orally administered form, the adoptive immunity cells may be prepared into the adoptive immunity cell composition with an excipient that is pharmaceutically acceptable, and that does not interfere with the activity of the adoptive immunity cells. Examples of such excipients include starch, mannitol, lactose, magnesium stearate, cellulose, polymerized amino acid, and albumin.

When the adoptive immunity cell composition of the present invention is a parenterally administered form, the adoptive immunity cells may be prepared into an adoptive immunity cell with a carrier that is pharmaceutically acceptable, and that does not interfere with the activity of the adoptive immunity cells. Examples of such excipients include, water, common salts, dextrose, ethanol, glycerol, and DMSO.

### Cancer Preventing, Cancer Treating, Cancer Metastasis Suppressing, or Cancer Recurrence Suppressing Agent

The cancer preventing, cancer treating, cancer metastasis suppressing, or cancer recurrence suppressing agent of the present invention contains a compound that inhibits the expression or activity of DDX3X.

Examples of the compound that inhibits DDX3X expression include a polynucleotide that comprises a base sequence (hereinafter, also referred to simply as “antisense sequence”) complementary to the sequence (hereinafter, also referred to simply as “target sequence”) in the whole region or a part of the region of the sense strand of DDX3X gene.

Examples of such polynucleotides include antisense nucleotides, siRNA (small interfering RNA), and shRNA (small hairpin RNA).

The target sequence can be determined by performing an NCBI BLAST search. Preferably, the target sequence is selected from the exon regions of the DDX3X gene. Preferably, the target sequence is highly specific to the target DDX3X gene sequence.

The target sequence is, for example, 15 to 30 bases long, preferably 18 to 25 bases long, more preferably 18 to 25 bases long, further preferably 19 to 23 bases long, particularly preferably 19 to 21 bases long.

The antisense nucleotide may be RNA or DNA. Further, the antisense nucleotide may have a sequence with one to several bases (e.g., 1 to 2 bases, 1 to 3 bases, 1 to 5 bases) attached to at least one of the terminals of the antisense sequence, or with the deletion, substitution, or addition of one to several bases within the antisense sequence, provided that the antisense nucleotide has the effect to suppress DDX3X gene expression.

For example, a double-stranded polynucleotide that consists of a polynucleotide comprising the target sequence (sense strand), and a polynucleotide comprising the antisense sequence (antisense strand) may be used as the siRNA.

The sense strand and the antisense strand may be longer than the target sequence by one or several bases (e.g., 1 to 2 bases, 1 to 3 bases, 1 to 5 bases), and may have, for example, two uracil (U) bases added to the terminal (preferably, the 3′ end). Further, the antisense strand and/or the sense strand may have a sequence with one to several bases, U, T, G, C, or A (e.g., 1 to 2 bases, 1 to 3 bases, 1 to 5 bases), attached to at least one of the terminals of the antisense sequence or the target sequence, or with the deletion, substitution, or addition of one to several such bases within the antisense sequence or the target sequence, provided that the antisense strand and the sense strand has the effect to suppress DDX3X gene expression.

Examples of the shRNA (small hairpin RNA) include those containing the siRNA sense and antisense strands joined to each other with a regulatory portion (loop portion), which may be a nucleotide sequence, a non-nucleotide sequence, or a combination of these.

When the regulatory portion is a nucleotide sequence, examples of the nucleotide sequence include a nucleotide sequence of at least one base and less than 10 kb, preferably a nucleotide sequence of one base to several hundred bases, further preferably a nucleotide sequence of one base to several ten bases, particularly preferably a nucleotide sequence of 1 to 20 bases, and a nucleotide sequence consisting of a sequence that can produce polynucleotides of the foregoing lengths in the cytoplasm by splicing or other cellular mechanisms. The nucleotide sequence forming the regulatory portion may include the sense sequence and the antisense sequence. Further, the nucleotide sequence forming the regulatory portion may be one of or a combination of two or more of the following sequences:

cytoplasmically oriented sequences, such as poly-A, tRNA, Usn RNA, and retrovirus-derived CTE sequence;

sequences having a decoy activity, such as NFκβ-binding sequence, E2F-binding sequence, SSRE, and NF-AT;

interferon induction suppressing sequences, such as adenovirus VA1 or VA2 RNA;

sequences having RNase suppressing activity, antisense activity, ribozyme activity, and the like;

marker sequences specifying tRNA or expression sites; and

selection marker sequences for detection with Escherichia coli.

The functional sequences requiring a partial double strand for decoy activity and the like may be produced with a complementary nucleotide. The regulatory portion may be designed to include a sequence required for splicing an intron donor sequence and acceptor sequence, allowing a part of the regulatory portion sequence to be cut and rejoined in cells having the splicing mechanism. The regulatory portion sequence configured above more desirably improves the RNA function suppressing effect, and stabilizes the sense sequence and the antisense sequence.

When the regulatory portion is a non-nucleotide sequence, specific examples include PNA (peptide nucleic acid), a chemically synthesized analog with the polyamide backbone, similar to nucleic acid.

In the present invention, a decoy nucleic acid against DDX3X gene for suppressing DDX3X gene transcription also may be used to suppress DDX3X gene expression.

Examples of the known compounds that inhibit DDX3X activity include the compounds described in WO2011/039735, specifically compounds represented by the following formulae.

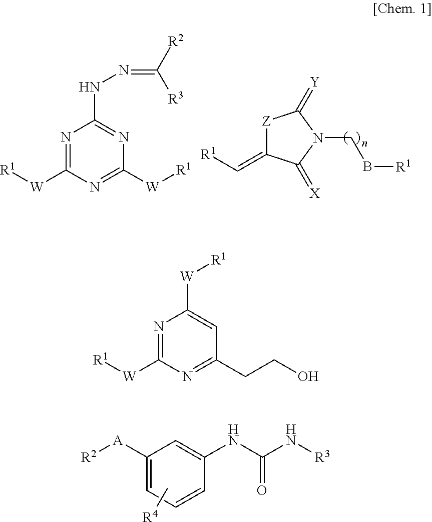

[wherein,

Z represents CH₂ or S,

X and Y independently represent 0 or S,

n ranges from 0 to 4,

B does not exist, or represents

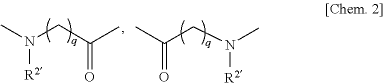

(wherein q ranges from 0 to 4, and R^{2′} represents hydrogen, —(CH₂)_{w′}—OH, or —(CH₂)_{w′}—NH₂ (where w′ is an integer of 1 to 3)), or B is C═O,

R¹, R², and R³ are each independently selected from the group consisting of H, a linear or branched alkyl group of 1 to 6 carbon atoms, an unsubstituted or substituted phenyl group, an unsubstituted or substituted phenylalkenyl group, an unsubstituted or substituted phenylalkynyl group, an unsubstituted or substituted biphenylalkyl group, an unsubstituted or substituted heterocyclic group, an unsubstituted or substituted polycyclic group, an unsubstituted or substituted alicyclic group, or (R^{1a}—)ₘ(L-)ₚR^{1b}— (wherein R^{1a} and R^{1b} may be the same or different, and represent an unsubstituted or substituted heterocyclic group or an unsubstituted or substituted phenyl group, R^{1a} also represents an unsubstituted or substituted polycyclic group, L represents a bivalent linking group selected from the group consisting of —(CH₂)_{q}—, —HC═CH—, —C≡C—, —C(═O)—, —O—, —S—, —S(═O)—, —S(═O)₂—, —NHCONH—, and —NR^{1c}—, where R^{1c} is hydrogen or alkyl, m and p each independently represent 0 or 1, and q is an integer of 1 to 3); or

R² and R³ may together form cycloalkyl, cycloalkenyl, a non-aromatic heterocyclic ring, or a condensed or polycyclic ring, or 2-oxyindole (the cycloalkyl, cycloalkenyl, condensed or polycyclic non-aromatic heterocyclic ring may be substituted with one or more substituents selected from the foregoing group),

W does not exist, or independently represents O, S, NH, NHCH₂, or N—R⁵ (where R⁵ is a linear or branched alkyl group of 1 to 6 carbon atoms),

A does not exist, or represents CONH, NHCO, or NHCONH,

R⁴ represents H, non-substituted or substituted alkyl of 1 to 6 carbon atoms, non-substituted or substituted alkenyl, non-substituted or substituted alkynyl, halogen, haloalkyl, COOH, OCH₃, NO₂, NH₂, CN, OZ′, or SZ′ (where Z′ is H, or non-substituted or substituted alkyl of 1 to 6 carbon atoms)].

Further examples of the known compounds that inhibit DDX3X activity include the compounds described in Bioorganic & Medicinal Chemistry Letters, Volume 22, Issue 5, 1 Mar. 2012, Pages 2094-2098, specifically compounds represented by the following formulae

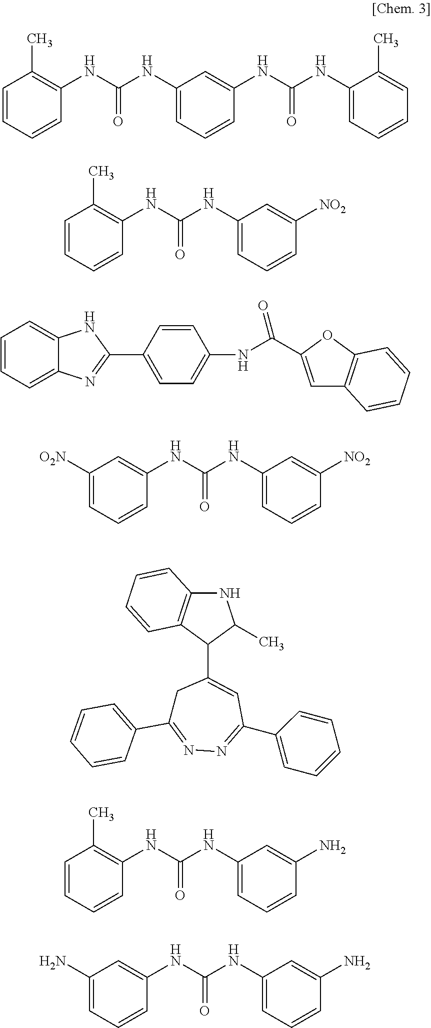

Further examples of the known compounds that inhibit DDX3X activity include the following compounds.

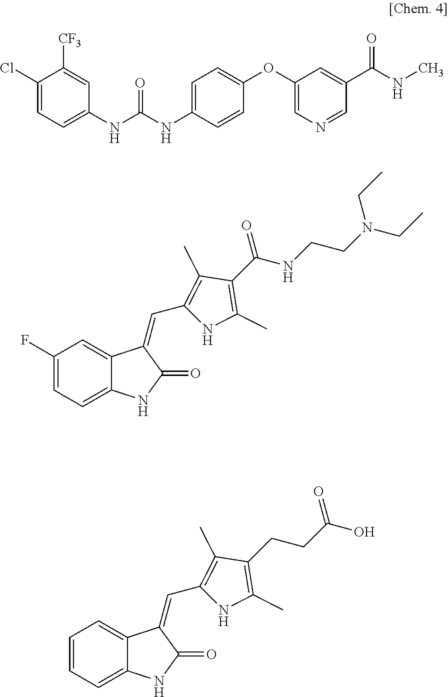 [SMILES: CNC(=O)c1cncc(Oc2ccc(NC(=O)Nc3ccc(Cl)c(C(F)(F)F)c3)cc2)c1 ; CCN(CC)CCNC(=O)c1c(C)[nH]c(/C=C2\C(=O)Nc3ccc(F)cc32)c1C ; Cc1[nH]c(/C=C2\C(=O)Nc3ccccc32)c(C)c1CCC(=O)O]

These compounds may be in the form of pharmaceutically acceptable salts.

Inhibition of the helicase DDX3X reduces the following four miRNAs: miRNA:hsa-mir-301a, hsa-mir-301b, hsa-mir-429, and hsa-miR-3922. It can therefore be said that inhibiting the activity of one or more of (preferably all of) these miRNAs is essentially the same as inhibiting the DDX3X activity. Accordingly, compounds that inhibit miRNA activity fall within the compounds that inhibit DDX3X activity according to the present invention.

The base sequences of these miRNAs are as follows.

hsa-mir-301a
(miRBase accession number MI0000745):
(SEQ ID NO: 93)
ACUGCUAACGAAUGCUCUGACUUUAUUGCACUACUGUACUUUACAGC
UAGCAGUGCAAUAGUAUUGUCAAAGCAUCUGAAAGCAGG
hsa-mir-301b
(miRBase accession number MI0005568):
(SEQ ID NO: 94)
GCCGCAGGUGCUCUGACGAGGUUGCACUACUGUGCUCUGAGAAGCAG
UGCAAUGAUAUUGUCAAAGCAUCUGGGACCA
hsa-mir-429
(miRBase accession number MI0001641):
(SEQ ID NO: 95)
CGCCGGCCGAUGGGCGUCUUACCAGACAUGGUUAGACCUGGCCCUCU
GUCUAAUACUGUCUGGUAAAACCGUCCAUCCGCUGC
hsa-miR-3922
(miRBase accession number MI0016429):
(SEQ ID NO: 96)
GGAAGAGUCAAGUCAAGGCCAGAGGUCCCACAGCAGGGCUGGAAAGC
ACACCUGUGGGACUUCUGGCCUUGACUUGACUCUUUC

Examples of the compounds that inhibit miRNA activity include polynucleotides that include a base sequence (antisense miRNA sequence) complementary to a part of or the entire region of miRNA (hereinafter, also referred to simply as “target sequence”).

Examples of the polynucleotides include antisense nucleotides.

The target sequence is, for example, 10 to 30 bases long, preferably 10 to 20 bases long, more preferably 12 to 18 bases long, further preferably 14 to 16 bases long.

The antisense nucleotide is, for example, RNA, DNA, or LNA. The antisense nucleotide may have a sequence with one to several bases (e.g., 1 to 2 bases, 1 to 3 bases, 1 to 5 bases) attached to at least one of the terminals of the antisense miRNA sequence, or with the deletion, substitution, or addition of one to several bases within the antisense miRNA sequence, provided that the antisense nucleotide has the effect to suppress miRNA activity.

### Examples

The present invention is described below in greater detail using Examples. It should be noted that the present invention is in no way limited by the following descriptions.

The materials and methods used in Examples are as follows.

### Materials and Methods

### Mice

Female C57BL/6J (B6) mice were purchased from CLEA Japan, maintained in a pathogen-free environment, and used for experiments at the age of 8-10 weeks.

All animal experiments were approved by the Niigata University Ethics Committee for Animal Experiments.

### Tumor Cells

B16F10, a melanoma of B6 origin, was maintained in vitro. Parental tumor cells were labeled with phycoerythrin (PE)-conjugated, anti-CD133 monoclonal antibodies (13A4) and anti-PE microbeads (Miltenyi Biotec). CD133-positive and CD133-negative tumor cells were isolated using autoMACS(trade name) (Miltenyi Biotec) according to the manufacturer's protocol. The cell purity was more than 90%.

### Monoclonal Antibodies and Flow Cytometry

Hybridomas producing monoclonal antibodies against murine CD4 (GK1.5, L3T4), CD8 (2.43, Lyt-2), CD3 (2C11), and murine CD62L (MEL14) were obtained from the American Type Culture Collection. Anti-CD4 monoclonal antibodies, anti-CD8 monoclonal antibodies, and anti-CD62L monoclonal antibodies were produced as ascites fluid from sub-lethally irradiated (500 cGy) DBA/2 mice. PE-conjugated anti-CD80 (16-10A), anti-CD86 (GL1), anti-CD62L (MEL14), anti-CD8 (2.43), and anti-CD25 (PC61) monoclonal antibodies; fluorescein isothiocyanate (FITC)-conjugated anti-Thy1.2 (30-H12) monoclonal antibodies; and anti-CD4 (GK1.5) monoclonal antibodies were purchased from BD PharMingen. Analyses of cell-surface phenotypes were conducted through direct immunostaining of 0.5 to 1×10⁶ cells with conjugated antibodies. In each sample, a total of 10,000 cells were analyzed using a FACScan(trade name) flow microfluorometer (Becton Dickinson). PE-conjugated subclass-matched antibodies used as isotype controls were also purchased from BD PharMingen. The samples were analyzed with CellQuest(trade name) software (Becton Dickinson).

### Fractionation of T Cells

T cells in the lymph node (LN) cell suspension were concentrated by passage through nylon wool columns (Wako Pure Chemical Industries). To yield highly purified (>90%) cells with down-regulated CD62L expression (CD62L^{low}), LN T cells were further isolated by a panning technique using T-25 flasks pre-coated with goat anti-rat immunoglobulin antibody (Ig Ab) (Jackson ImmunoResearch Laboratories)/anti-CD62L (MEL14) monoclonal antibody, and by a magnetic bead technique using sheep anti-rat-Ig Ab/anti-CD62L monoclonal antibody-coated DynaBeads M-450 (Dynal). In some experiments, cells were further separated into CD4-negative and CD8-negative cells by depletion using magnetic beads, as described in Hiura T, Kagamu H, Miura S, Ishida A, Tanaka H, Tanaka J, et al., Both regulatory T cells and antitumor effector T cells are primed in the same draining lymph nodes during tumor progression. J Immunol. 2005; 175: 5058-66. For the purification purpose, highly purified CD4-positive cells were obtained by positive selection using anti-CD4 monoclonal antibody-coated Dynabeads and Detachabeads (Invitrogen).

### Bone Marrow-Derived Dendritic Cells

Dendritic cells (DCs) were generated from bone marrow cells (BMs) according to the method described in Fujita N, Kagamu H, Yoshizawa H, Itoh K, Kuriyama H, Matsumoto N, et al. CD40 ligand promotes priming of fully potent antitumor CD4(+) T cells in draining lymph nodes in the presence of apoptotic tumor cells, J Immunol. 2001; 167: 5678-88. In brief, BMs obtained from the femurs and tibias of mice were placed in T-75 flasks for 2 hours at 37° C. in complete medium (CM) containing 10 ng/ml of recombinant murine granulocyte-macrophage colony-stimulating factor (rmGM-CSF, a gift from KIRIN). Non-adherent cells were isolated, and cultured in fresh flasks. On day 6, non-adherent cells were harvested by gentle pipetting. CM consisted of RPMI 1640 medium supplemented with 10% inactivated lipopolysaccharide (LPS) qualified (endotoxin-free) fetal calf serum, 0.1 mM nonessential amino acids, 1 μM sodium pyruvate, 100 U/mL of penicillin, 100 μg/mL of streptomycin sulfate (all from Life Technologies, Inc.), and 5×10⁻⁵ M 2-mercaptoethanol (Sigma Chemical).

### DC/Tumor Vaccine-Draining LN Cells

BMs and DCs were co-cultured in CM overnight with the same number of irradiated tumor cells (5,000 cGy). B6 mice were inoculated s.c. with 1×10⁶ BM-DC and tumor cells in both flanks. Inguinal LNs draining BM-DC and tumor vaccines were harvested. Single-cell suspensions were prepared according to the method described in Watanabe S, Kagamu H, Yoshizawa H, Fujita N, Tanaka H, Tanaka J, et al., The duration of signaling through CD40 directs biological ability of dendritic cells to induce antitumor immunity. J Immunol. 2003; 171: 5828-36.

### Adoptive Immunotherapy

B6 mice were injected s.c. in the midline with B16-F10 tumor cells suspended in 100 μl of Hanks' balanced salt solution (HBSS) to establish a subcutaneous tumor model. Two or three days after the inoculation, the mice were sub-lethally irradiated (500 cGy) and then infused i.v. with T cells isolated from BM-DC/tumor vaccine-draining lymph nodes. These LN cells were stimulated with anti-CD3 monoclonal antibodies (2C11) and cultured in CM containing 40 U/mL of IL-2 for 3 days to obtain a sufficient number of cells, as described in Fujita N, Kagamu H, Yoshizawa H, Itoh K, Kuriyama H, Matsumoto N, et al. CD40 ligand promotes priming of fully potent antitumor CD4(+) T cells in draining lymph nodes in the presence of apoptotic tumor cells. J Immunol. 2001; 167: 5678-88. The perpendicular diameters of subcutaneous tumors were measured using calipers.

### Cytokine ELISAs

T cells were stimulated with immobilized anti-CD3 monoclonal antibodies or antigen-pulsed BM-DCs in CM. Supernatants were harvested and assayed for IFN-γ, IL-4, and IL-17 content by a quantitative “sandwich” enzyme immunoassay using a murine IFN-γ, IL-4, and IL-17 ELISA kit (Genzyme), according to the manufacturer's protocol.

### In Vitro Proliferation Assay

Melanoma cells were labeled with 5 μM 5-(6)-carboxyfluorescein diacetate succinimidyl diester (CFSE; Molecular Probes) in HBSS at 37° C. for 15 min and washed twice before CD3 stimulation. The ratio of CFSE-labeled tumor cells to unlabeled tumor cells was 1:10. The tumor cells were cultured in CM at 1×10⁵/mL, counted, and analyzed using a microfluorometer to determine the number of CFSE-labeled cells.

### Immunoblotting Assay

Cells were harvested and lysed in Nonidet P-40 buffer containing a protease-inhibitor mixture (Sigma). Equal microgram amounts of proteins were subjected to SDS-7.5% PAGE and transferred to polyvinylidene difluoride membrane (Millipore). Immunoblots from tumor cells were probed with antibodies against DDX3X (Sigma) and β-actin (Sigma). Secondary antibodies consisted of anti-mouse Ig and anti-rabbit Ig conjugated to horseradish peroxidase (HRP; Bio Rad, Dako). Immunoreactive protein bands were visualized using the ECL kit (Pierce). At least, three independent experiments were performed for all analyses.

Knockdown of DDX3X by shRNA

Knockdown of DDX3X was obtained using an shRNA lentiviral (pLK0.1-puro) plasmid (Sigma Aldrich). The oligonucleotides containing the DDX3X target sequence that were used were CCGGACGTTCTAAGAGCAGTCGATTCTCGAGAATCGACTGCTCTTAGAACGTTTTTTG (SEQ ID NO: 97). B16 CD133-positive cells were added in fresh media, and hexadimethrine bromide (8 μg/ml) was added to each well. The cells were co-transfected with the pLK0.1-puro plasmid plus the packaging vector according to the manufacture's protocol. The media were changed approximately 16 hours after transfection, and the cells were cultured for an additional 48-72 hours. Experimental cells were incubated with the fresh media containing puromycin (2.0 μg/ml), and the media were replaced with fresh puromycin (2.0 μg/ml)-containing media every 3-4 days until resistant colonies could be identified. A minimum of five puromycin-resistant colonies were picked and each clone was expanded for the assay. The efficiency of DDX3X knockdown was determined by immunoblotting.

### Statistical Analysis

Comparison between groups was performed using Student's t-test. Dynamic tumor-growth data was analyzed by a multivariate, general linear model. Differences were considered significant for P<0.05. Statistical analysis was performed with SPSS statistical software (SPSS) or GraphPad Prism 5.0 software (GraphPad Software).

### DDX3X and Partial Peptides Thereof

The DDX3X and partial peptides thereof used were prepared by chemical synthesis.

DDX3X has the amino acid sequence represented by SEQ ID NO: 1.

Peptide J has the amino acid sequence represented by SEQ ID NO: 89.

Peptide K has the amino acid sequence represented by SEQ ID NO: 88.

DDX3X-10 mer has the amino acid sequence represented by SEQ ID NO: 17.

DDX3X-15 mer has the amino acid sequence represented by SEQ ID NO: 90.

DDX3X-20 mer has the amino acid sequence represented by SEQ ID NO: 91.

### Example 1

### Release of CD133-Positive Tumor Antigen-Specific Cytokine from DDX3X-Specific CD4-Positive T Cells

The inventors investigated whether T cells primed with synthesized DDX3X antigen could recognize CD133-positive melanoma cells. To test this, T cells with down-regulated expression of CD62L (CD62L^{low}) were isolated from lymph nodes (LN) draining DC that were pulsed with synthesized DDX3X.

The CD62L^{low} CD4-positive or CD8-positive T cells (1×10⁵ cells) isolated from the lymph nodes were stimulated with 1×10⁴ dendritic cells in 200 μl of complete medium (CM) for 48 hours in a 96-well plate. The dendritic cells used for stimulation were stimulated overnight with the same number of irradiated CD133-positive tumor cells or CD133-negative tumor cells (5,000 cGy), or synthesized DDX3X (5 μg/ml). The dendritic cells were purified with CD11c microbeads prior to co-culture.

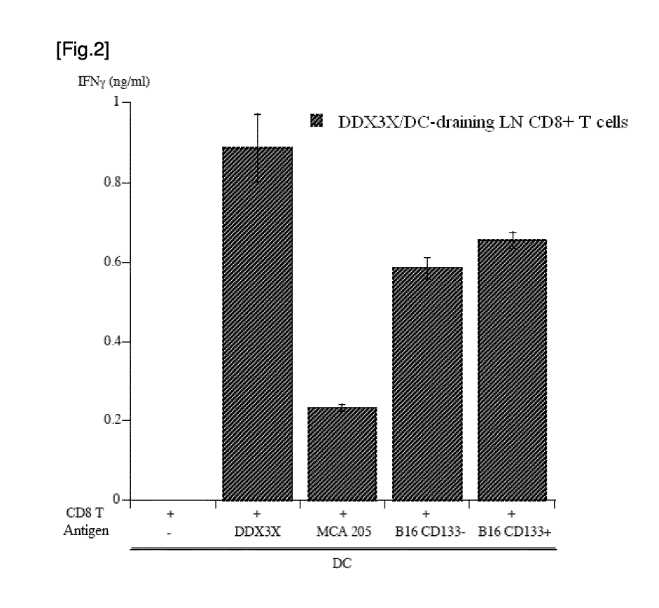

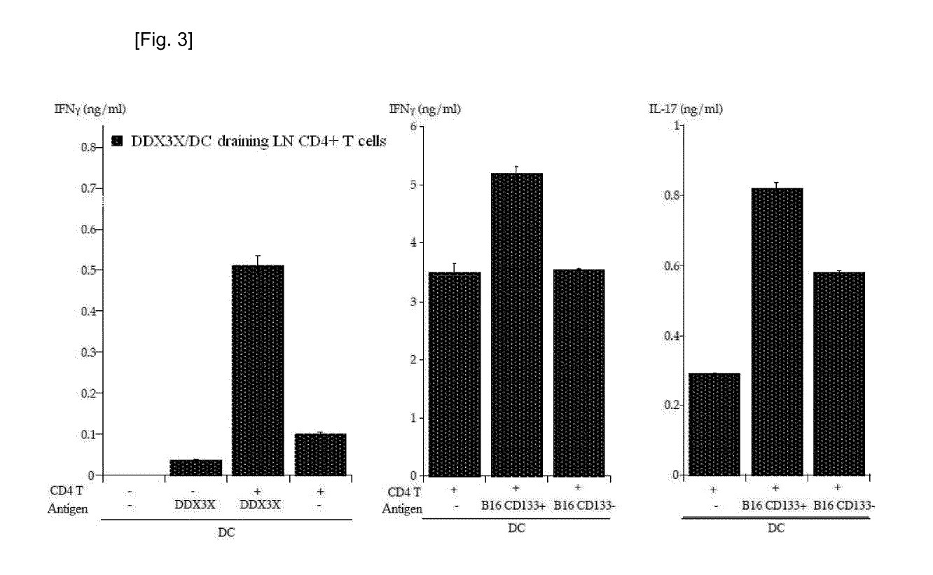

The inventors found that DDX3X-specific CD4-positive T cells thus obtained secreted IFN-γ and IL-17 in a melanoma CSC-specific manner. However, DDX3X-specific CD8-positive T cells responded to both CD133-negative and CD133-positive melanoma cells (FIGS. 2 and 3).

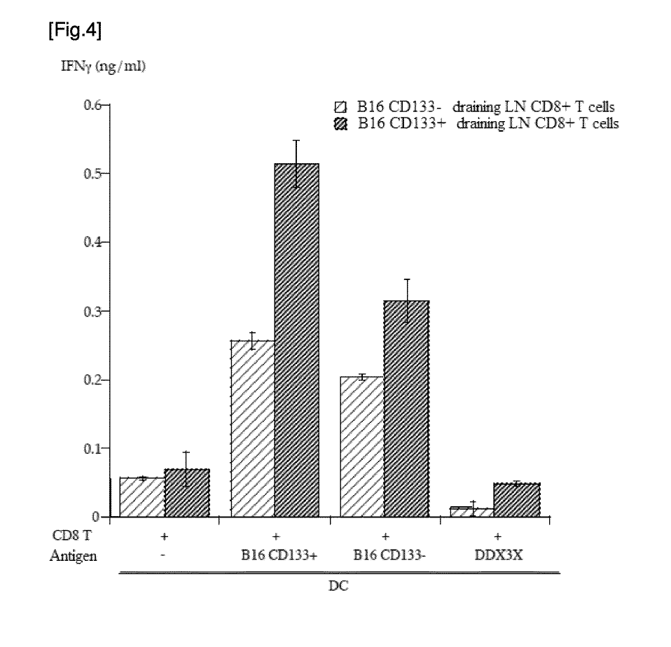

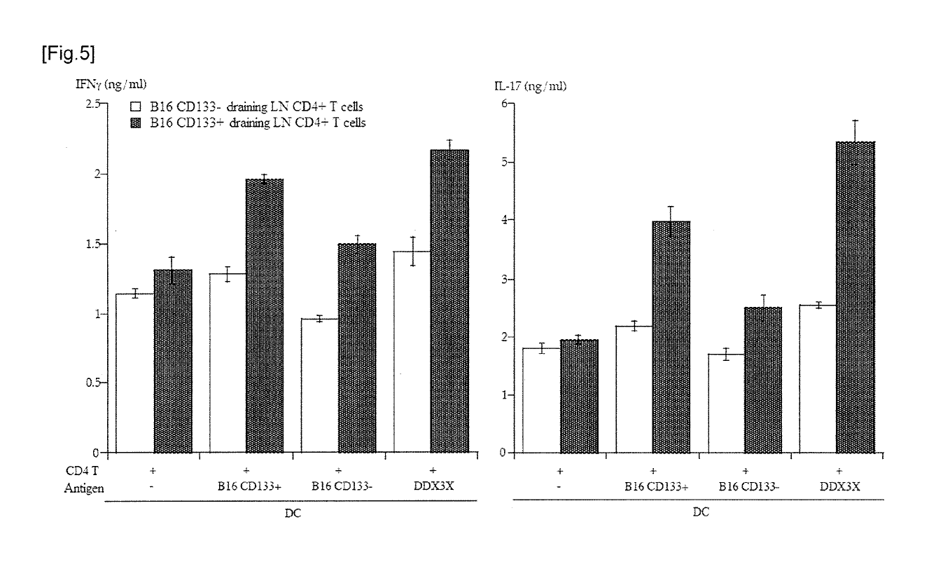

Next, the inventors tested whether melanoma CSC-specific T cells recognized DDX3X and produced cytokines. It was found that melanoma CSC-specific CD4-positive T cells primed with vaccinated CD133-positive melanoma cells produced cytokines in a DDX3X-specific manner (FIGS. 4 and 5). Surprisingly, the melanoma CSC-specific CD4-positive T cells produced even more cytokines upon DDX3X stimulation than upon stimulation with the melanoma CSC itself.

The DDX3X-specific CD4-positive T cells were thus found to have anti-tumor activity against DDX3X-expressing tumor cells, and can be used for adoptive immunotherapy.

### Example 2

### Vaccination with DDX3X Induced Protective Immunity Against Melanoma Cells

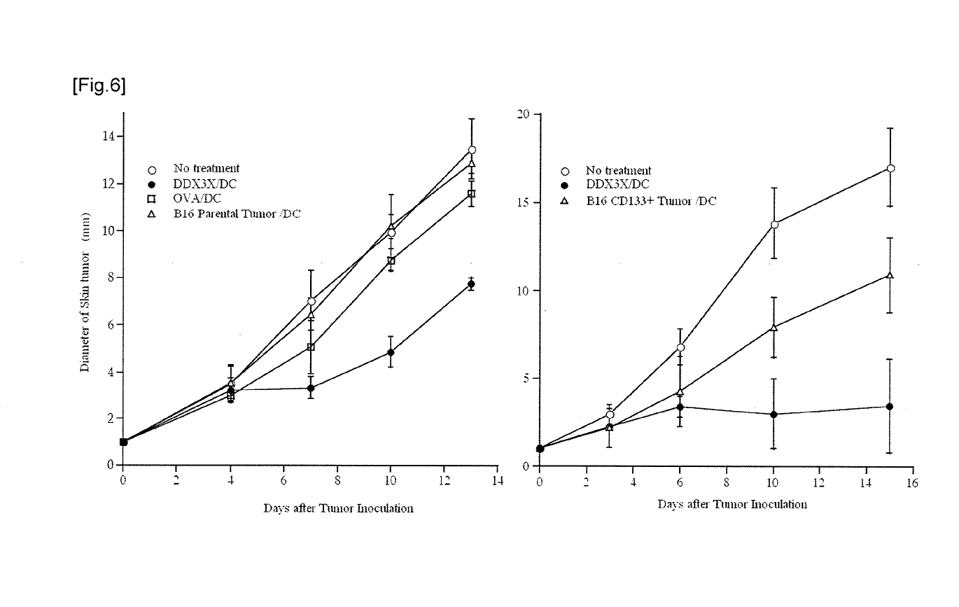

To examine whether protective immunity against B16 melanoma cells could be induced by vaccination with synthesized DDX3X, dendritic cells pulsed with DDX3X or ovalbumin (OVA) at 5 μg/ml, or co-cultured with irradiated CD133-positive tumor cells (5,000 cGy) for 8 hours, were subcutaneously (s.c.) injected in the right flank of the mice. Fourteen days later, the mice were s.c. inoculated in the midline of the abdomen with 2×10⁶ melanoma cells. Each group contained 5 mice. As shown in FIG. 6, tumor growth was significantly more suppressed in the mice that received the DDX3X-pulsed dendritic cells vaccination compared to the mice that received either no treatment or treatment with OVA-pulsed DCs. Furthermore, the mice vaccinated with DCs pulsed with DDX3X exhibited significantly more potent protective immunity than did mice injected with dendritic cells co-cultured with irradiated CD133-positive tumor cells.

### Example 3

### Vaccination with DDX3X Exhibited Therapeutic Efficacy Against Established Skin Tumors

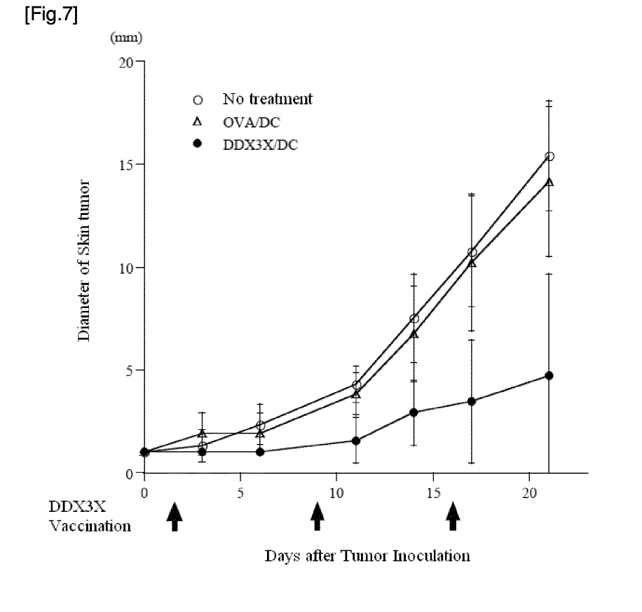

The inventors further tested if vaccination with DDX3X had therapeutic efficacy against established tumors. On days 2, 9, and 16 after s.c. inoculation of 1×10⁶ B16 melanoma cells in the midline of the abdomen, 1×10⁶ dendritic cells were injected in the right flank. DDX3X- or OVA-pulsed 1×10⁶ dendritic cells at 5 μg/ml were injected s.c. in the right flank. Each group contained 12 mice. FIG. 7 represents the tumor growth curve of each mouse. It was found that 6 of 12 mice vaccinated with DDX3X-pulsed dendritic cells were eventually cured. In the other DDX3X-pulsed, dendritic cells-vaccinated mice, skin-tumor growth was significantly suppressed. All the mice that received no treatment or were vaccinated with OVA-pulsed DC died of the tumor.

### Example 4

### Significance of DDX3X for Immunogenicity of CD133-Positive Melanoma

It has been shown previously that B16 melanoma cells possess a number of immunogenic proteins.

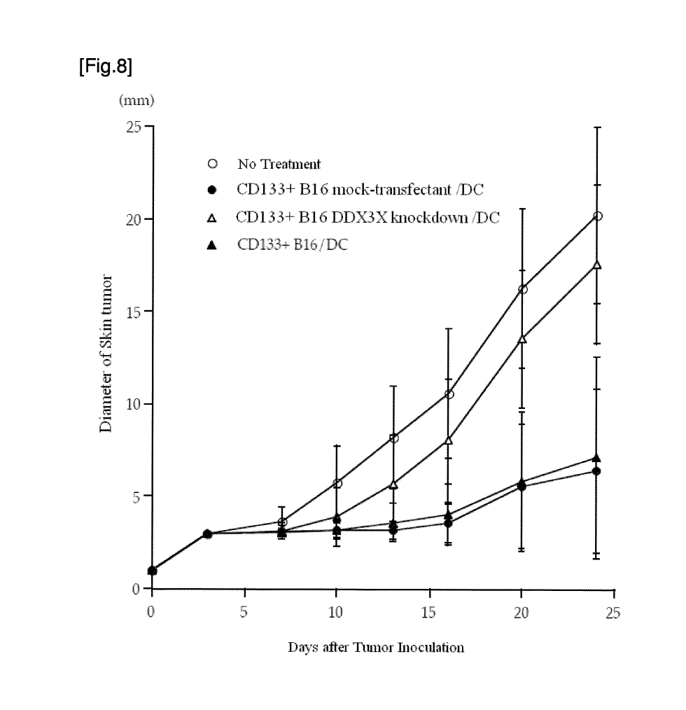

To elucidate the significance of DDX3X for the immunogenicity of DDX3X, the inventors established CD133-positive melanoma cells (CD133-positive B16 cells lacking DDX3X) by knockdown of DDX3X using shRNA. A total of 5,000 cGy-irradiated mock-shRNA and DDX3X knockdown CD133-positive B16 cells were co-cultured with dendritic cells (DCs) for 8 hours. One million CD11c-positive cells purified with CD11c microbeads and autoMACS(trade name) were subcutaneously administered to B6 mice. Two weeks after immunization, mice were subcutaneously inoculated along the midline of the abdomen with 2×10⁶ B16 melanoma cells. Each group contained 5 mice. As shown in FIG. 8, mice vaccinated with CD133-positive parental cells, or CD133-positive mock-transfectant tumor cells (control) had effective protective immunity. In contrast, vaccination with CD133-positive tumor cells lacking DDX3X failed to induce antitumor protective immunity. In other words, the CD133-positive melanoma lacking DDX3X lost the vaccine effect.

### Example 5

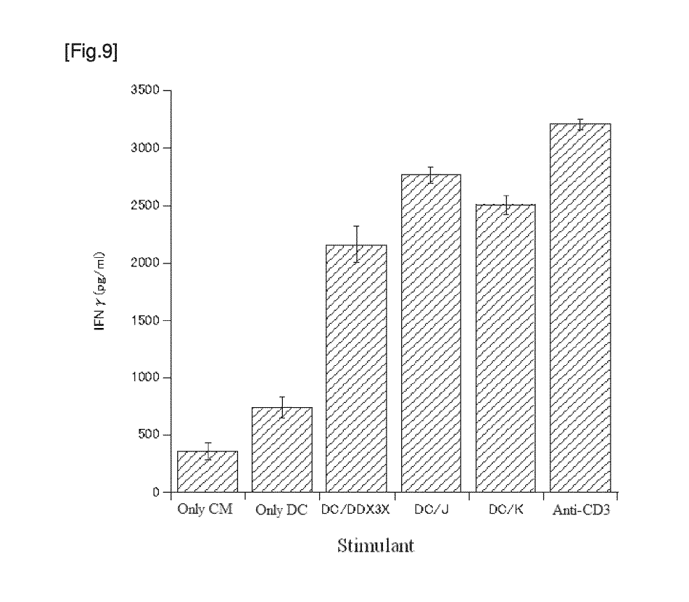

Peripheral blood (15 ml) was collected from a small-cell lung cancer patient. A mononulclear cell fraction was collected by density-gradient centrifugation using Lymphoprep(trade name) (Cosmo Bio), and CD14⁺ cells were isolated with CD14 microbeads and autoMACS. The CD14⁺ cells were cultured with rhGM-CSF (1 ng/ml, a gift from Kirin) and IL-4 (10 ng/ml, R&D systems), and used by day 5 after being differentiated and matured into dendritic cells. The dendritic cells were cultured overnight in medium containing synthesized DDX3X protein (3.3 μg/ml) or the same concentration of peptides (peptide J, peptide K), and CD11c-positive cells purified with CD11c microbeads and autoMACS were used as antigen-presenting cells. In order to remove naive T cells and regulatory T cells from the CD14 fraction cells, CD62L^{high} cells were removed by using anti-human CD62L antibody (1H3)-conjugated Dynabeads. The CD62L^{low} CD14⁻ cells were cultured for 48 hours on a BD BioCoat T cell activation plate (Becton Dickinson), and cultured for 4 days in medium containing 20 U/ml of rhIL-2 (a gift from Shionogi). FACS confirmed that 95% or more of the cells that increased about 10 fold were CD3⁺ T cells, and the CD3⁺ T cells were used as responding cells. The responding cells (1×10⁵) and antigen-presenting cells (1×10⁴) were cultured for 24 hours in a 200-μl medium in a round-bottom 96-well plate, and the IFN-γ concentration of the collected supernatant was measured by ELISA. A supernatant from a culture of 1×10⁵ responder cells incubated in an anti-CD3 antibody-immobilized 96-well plate was used as a positive control. The results are presented in FIG. 9.

### Example 6

### CTL Induction and IFN-γ Production with DDX3X-Derived Peptide

DDX3X-specific CTL induction, and IFN-γ production by stimulation with DDX3X-derived peptides were evaluated.

Table 1 lists the reagents used. Table 2 is a list of the peptides used for induction.

TABLE 1

| Reagent | Supplier | Catalog number |
|---|---|---|
| Lymphoprep | AXIS SHIELD | 1114547 |
| Hank's Balanced | SIGMA | H9269-500ML |
| Salt Solution | | |
| heparin sodium injection | Ajinomoto | 70111 |
| AIM-V | Life Technologies | 12055-091 |
| Human AB Serum | Dainippon Sumitomo | 2931949 |
| Recombinant Human IL-7 | PeproTech, Inc. | 200-07 |
| Recombinant Human IL-2 | PeproTech, Inc. | 200-02 |
| Cell Banker | Wako Pure Chemical | 630-01601 |
| | Industries | |
| OptEIA Kit (Human IFN-γ) | BD Bioscience | 555142 |
| BD OptEIA Reagent Set B | BD Bioscience | 550534 |

TABLE 2

| Peptide | Sequence | Supplier |
|---|---|---|
| DDX3X-10 mer | LEDFLYHEGY | American Pep- |
| | | tide Co. Inc. |
| DDX3X-15 mer | KGADSLEDFLYHEGY | |
| DDX3X-20 mer | FVETKKGADSLE | |
| | DFLYHEGY | |

### Medium Preparation

Human AB serum was inactivated at 56° C. for 30 min, and filtered through a 0.22-μm filter (Serum Acrodisc, Pall). The inactivated human AB serum (50 mL) was added and mixed with 500 mL of AIM-V in a clean bench to prepare a medium. Heparin-containing HBSS was prepared by adding and mixing 10 mL of heparin sodium injection (10000 U/mL) with 500 mL of Hank's Balanced Salt Solution (20 U/mL heparin). These were stored at 4° C. until use.

### Screening of Blood Donor

Healthy volunteers were screened for individuals with HCT116 HLA-A0201, or HLA-A*2601, predicted to show high affinity to DDX3X peptides by software calculations (decamers were selected). Out of these volunteers, six had HLA-A*0201, and five had HLA-A*2601.

### Preparation of Peripheral Blood-Derived Mononulclear Cells (PBMC)

Peripheral blood (40 mL) obtained from healthy volunteers was diluted with heparin-containing HBSS (13 mL of HBSS per 20 mL of blood), and layered in a lymphocyte separation tube (Leucosep(trade name); Greiner) charged with 15 mL of Lymphoprep. After centrifugation (2,000 rpm, 20° C., 20 min), the middle layer (PBMC) was collected into a 50-mL centrifuge tube, and recentrifuged (1,800 rpm, 20° C., 5 min) after being diluted two times with heparin-containing HBSS. The resulting pellet was suspended in 10 mL of heparin-containing HBSS, and centrifuged (1,200 rpm, 4° C., 5 min). This procedure was repeated. The resulting PBMC pellet was suspended in medium (1 mL), and 1.5×10⁷ cells were used for DDX3X-specific CTL induction, whereas the remaining cells were used for antigen-presenting cells in re-stimulation. The PBMC used for antigen-presenting cells in re-stimulation were suspended in a Cell Banker and cryopreserved at −80° C., and thawed before use.

### Induction of DDX3X-Specific CTL

PBMC (1.5×10⁷ cells) were inoculated in a 24-well plate in 1.5×10⁶ cells/well (Day 0). Then, three types of DDX3X-derived peptides (final concentration 20 μg/mL each), and IL-7 (final concentration 10 ng/mL) were added to each well, and the cells were cultured at 37° C. in 5% CO₂.

The cells were re-stimulated after 1 week (Day 7). For re-stimulation, the PBMC cryopreserved as antigen-presenting cells in Day 0 were thawed, and the three types of DDX3X-derived peptides (final concentration 20 μg/mL each) were added and conjugated at 37° C. for 2 hours after adjusting the cells to 3×10⁶ cells/mL or less. This was followed by addition of a mitomycin C [Kyowa Hakko Kirin] solution to make the final concentration 50 μg/mL, and the cells were treated at 37° C. for 45 min. The cells were washed twice with AIM-V, and resuspended in medium to obtain an antigen-presenting cell suspension. After harvesting cells cultured for 1 week, the cells were inoculated in a 24-well plate in 1.2×10⁶ cells/well, and the antigen-presenting cell suspension was inoculated with the same number of cells. Finally, IL-7 was added at 10 ng/ml, and the cells were cultured at 37° C. in 5% CO₂. After 2 days (Day 9), half of the culture medium was gently removed from each well, and replaced with 40 U/mL of IL-2-containing medium for further culture. The half-volume replacement of the culture medium with 20 U/mL of IL-2-containing medium was repeated in the same fashion every other day (Day 11, Day 13). Re-stimulation was repeated in Day 14 and Day 21 using the same procedure. The cells were co-cultured in the presence of 20 U/mL of IL-2, and grown until Day 28 in culture medium replaced half with 20 U/mL of IL-2-containing medium every other day (Day 16, Day 18, Day 20, and Day 23, Day 25, Day 27).

### Evaluation of IFN-γ Production by DDX3X-Derived Peptide Stimulation

Cells harvested in Day 21 and Day 28 were appropriately diluted with medium, and inoculated in 96-well round-bottom plates (100 μL each). Then, a medium prepared to contain 40 μg/mL of DDX3X-derived peptide was added to 96-well round-bottom plates (100 μL each), and the cells were cultured in 5% CO₂/37° C. (peptide final concentration=20 μg/mL). ELISA was performed in triplicate for each stimulation. As a negative control, a solvent, DMSO, was used for stimulation.

Twenty-four hours after peptide stimulation, the culture supernatant was gently collected from each well. IFN-γ concentration in each culture supernatant was detected by ELISA, by using an ELISA reagent set (BD OptEIA ELISA set (human IFN-7)(trade name)) according to the manufacturer's protocol after modification. Specifically, coating antibodies, detection antibodies, and HRP-labeled antibodies were used after being diluted 500 times. Measurements were made with a visible wavelength absorbance microplate reader (VERSAmax(trade name); Molecular Device).

### Results

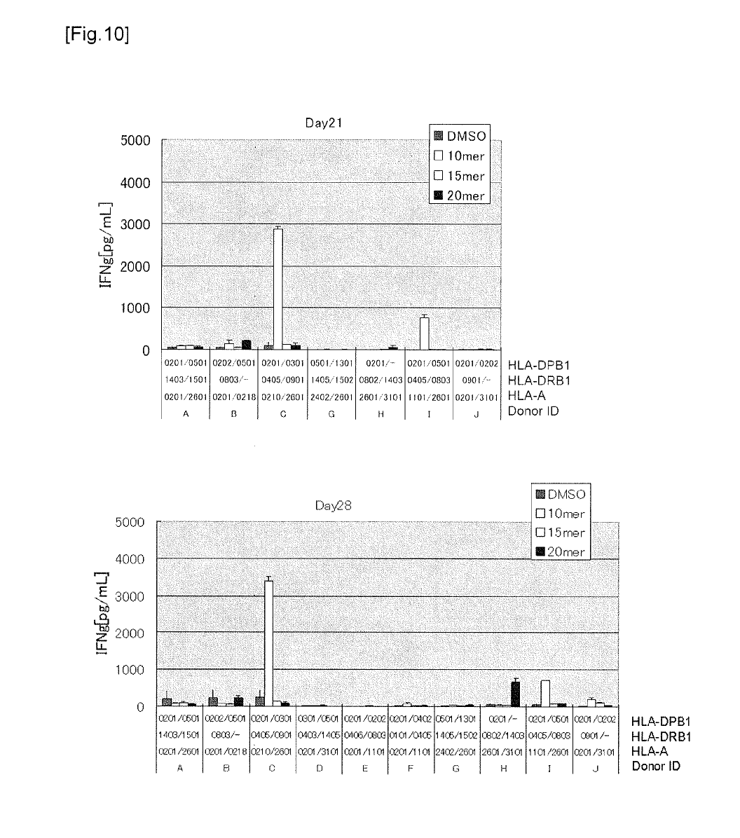

The results are presented in FIG. 10. In the Figure, the columns in the bar chart of each donor (A to J) represent DMSO, 10 mer, 15 mer, and 20 mer from the left.

In the evaluation at Day 21, no IFN-γ production was observed in any of the stimulations in the three samples with A*0201. On the other hand, two of the five samples with A*2601 showed IFN-γ production only in the DDX3X-10 mer stimulation.

In the evaluation at Day 28, one of the six samples with A*0201 showed IFN-γ production in the DDX3X-10 mer stimulation. On the other hand, IFN-γ production was observed in the DDX3X-20 mer stimulation in one of the five samples with A*2601 in addition to the two samples that showed IFN-γ production in the DDX3X-10 mer stimulation at Day 21.

These results demonstrate that stimulation specific induction of IFN-γ producing cells is possible by stimulation of healthy PBMC with DDX3X-derived peptides.

### Example 7

### DDX3X/DC-Immunostimulated CD4-Positive T Cells Had Anti-Tumor Effect

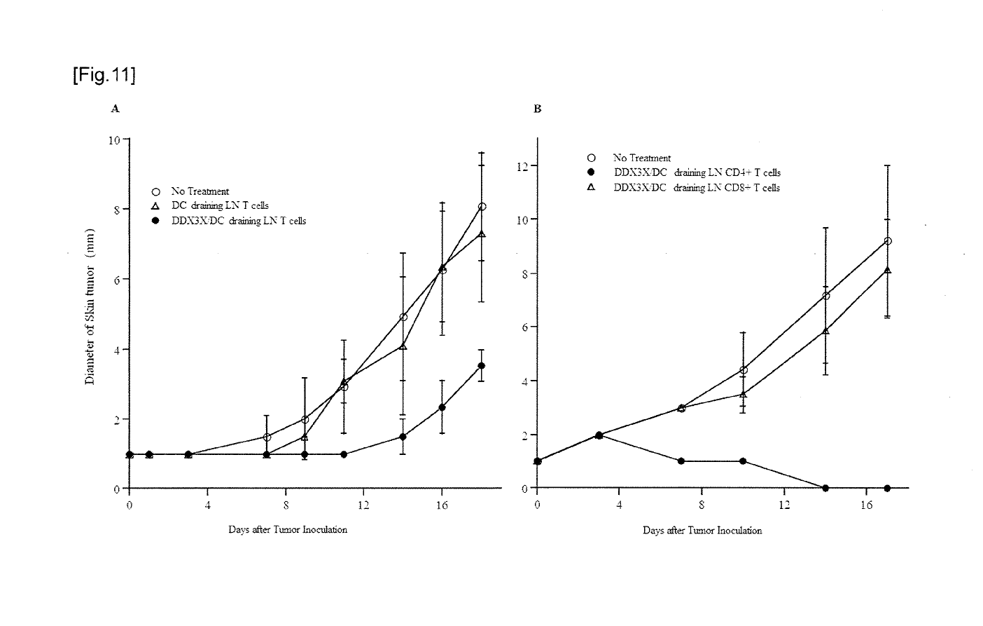

Dendritic cells were pulsed with synthesized DDX3X at 5 μg/mL for 8 hours and isolated as CD11c-positive cells (DDX3X/DC) using CD11c microbeads and autoMACS(trade name). CD62L^{low} T cells were isolated from lymph nodes draining DDX3X/DC vaccine. The CD62L^{low} T cells, which are lymph node T cells, were cultured for 5 days as described in the Materials and Methods. The cultured CD62L^{low} T cells were intravenously infused into the mice bearing 2-day established skin melanoma after sublethal whole body irradiation (500 cGy). The DDX3X-specific T cells were found to have anti-tumor activity, and greatly suppress skin tumor growth (FIG. 11A).

The DDX3X/DC vaccine-draining lymph node T cells were thus found to have anti-tumor therapeutic efficacy.

It was further investigated whether which of the CD4-positive T cells and the CD8-positive T cells were responsible for the anti-tumor activity. Lymph node T cells after a 5-day culture were further purified with magnetic beads to obtain CD4-positive T cells and CD8-positive T cells. 10×10⁶ CD4-positive lymph node T cells or CD8-positive T cells were infused intravenously. 10×10⁶ CD8-positive T cells were infused into the mice bearing 2-day established skin melanoma after sublethal whole body irradiation (500 cGy). However, no significant anti-tumor activity was recognized. On the other hand, the DDX3X-specific CD4-positive T cells showed high anti-tumor activity, and cured the tumor (FIG. 11B).

### Example 8

### Specific Expression of DDX3X

87.5 and S2 (human small cell lung cancer), HCT116 (human colon cancer), A549 (human non-small cell lung cancer), WM115 (human melanoma), and MCF7 (human breast cancer) cells were examined for expression of DDX3X in human tumor cells, using putative CSC markers CD133, CD44, and CD24.

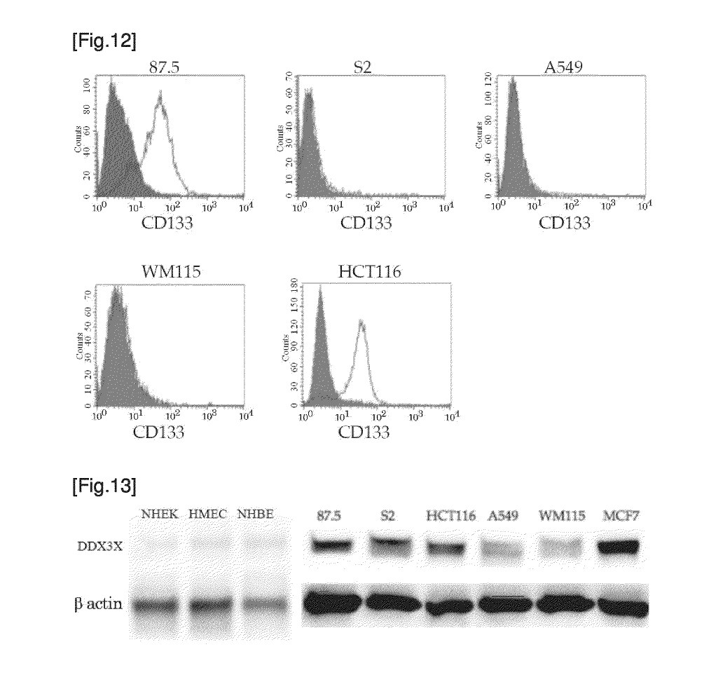

As shown in FIG. 12, the 87.5 and HCT116 cells expressed CD133, whereas other cancer cells did not. The 87.5 cells proliferated as floating aggregates and easily formed tumor spheres. MCF7 cells were found to exhibit the CD44⁺ and CD24^{-/low} phenotype, traditionally considered the breast cancer stem cell phenotype. (References 1 to 3 below).
Reference 1: Al-Hajj M, Wicha M S, Benito-Hernandez A, Morrison S J, Clarke M F. Prospective identification of tumorigenic thoracic cancer cells. Proceedings of the National Academy of
Sciences of the United States of America. 2003; 100: 3983-8.
Reference 2: Ponti D, Costa A, Zaffaroni N, Pratesi G, Petrangolini G, Coradini D, et al. Isolation and in vitro propagation of tumorigenic thoracic cancer cells with stem/progenitor cell properties. Cancer research. 2005; 65: 5506-11.
Reference 3: Kai K, Arima Y, Kamiya T, Saya H. Breast cancer stem cells. Breast cancer. 2010; 17: 80-5.

Whole cell lysates were extracted from normal human cells (human epidermal keratinocytes (NHEK), human microvascular endothelial cells (HMEC), normal human bronchial epithelial cells (NHBE)) and cancer cells (87.5, S2, HCT116, A549, WM115, MCF7). Immunoblots assay of tumor cells were conducted using antibodies against DDX3X and β-actin.

All of the examined cells expressed DDX3X, while normal human epidermal keratinocytes (NHEK), human microvascular endothelial cells (HMEC), and normal human bronchial epithelial cells (NHBE) faintly expressed DDX3X. Moreover, putative CSC marker positive cells, such as 87.5, HCT116, and MCF7, strongly expressed DDX3X (FIG. 13). Thus, it is likely that DDX3X is not only expressed in murine melanoma stem cells, but also expressed in various human tumors.

### Example 9

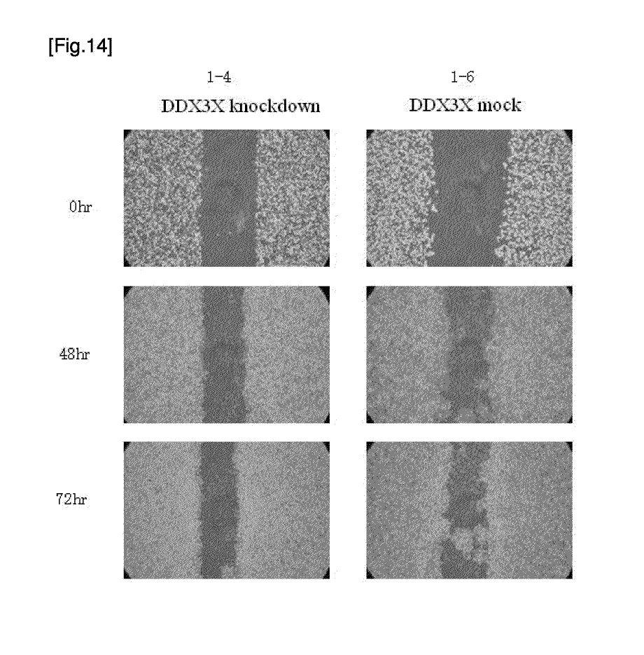

The human colon cancer cell line HCT116 is predominantly CD133-positive cells, and has highly expressed DDX3X. A cell injury repair experiment was conducted using 1-4 cells obtained by knockdown of DDX3X with shRNA introduced by using a lentiviral vector, and mock-transfectant 1-6 cells. It has been confirmed that the growth rates of the 1-4 cells and 1-6 cells are not different. The 1-4 cells and the 1-6 cells grown to subconfluence after simultaneous inoculation in a 24-well plate were linearly detached with a pipette tip, and the time course of injury repair was observed. The 1-4 cells with knockdown of DDX3X delayed the tissue repair (FIG. 14).

### Example 10

Given the high DDX3X expression in the small cell lung cancer cell line, an investigation was conducted for the presence of T lymphocytes that recognize DDX3X and produce cytokine in the peripheral blood of a small cell lung cancer patient. This experiment was approved by the Niigata University, School of Medicine, Ethics Committee.

Peripheral blood (15 ml) was collected after informed consent. A mononulclear cell fraction was collected by density-gradient centrifugation using Lymphoprep(trade name) (Cosmo Bio), and CD14⁺ cells were isolated with CD14 microbeads and autoMACS. The CD14⁺ cells were cultured with rhGM-CSF (1 ng/ml, a gift from Kirin) and rhIL-4 (10 ng/ml, R&D systems), and differentiated into dendritic cells by day 5. The dendritic cells were cultured overnight in medium containing synthesized DDX3X protein (3.3 μg/ml) or the same concentration of OVA, and CD11c-positive cells purified with CD11c microbeads and autoMACS were used as antigen-presenting cells. In order to remove naive T cells and regulatory T cells from the CD14 fraction cells, CD62L^{high} cells were removed by using anti-CD62L antibody (1H3)-conjugated Dynabeads. The CD62L^{low} CD14⁻ cells were cultured for 48 hours on a BD BioCoat(trade name) T cell activation plate (Becton Dickinson), and cultured for 4 days in medium containing 20 U/ml of rhIL-2 (a gift from Shionogi). FACS confirmed that 95% or more of the cells were CD3-positive T cells, and the CD3-positive T cells were used as responding cells. The responding cells (1×10⁵) and antigen-presenting cells (1×10⁴) were co-cultured for 24 hours in a 200-μl medium in a round-bottom 96-well plate, and the IFN-γ concentration was measured by ELISA using the supernatant collected after co-culture. Unpulsed dendritic cells, DDX3X-pulsed dendritic cells, and OVA-pulsed dendritic cells were used as the antigen-presenting cells. In Table 3, “Yes” means that IFN-γ production from T cells was confirmed with significant difference only in a co-culture with the DDX3X-pulsed dendritic cells. In the table, “% Treg” and “% Teff” represent the proportions of the regulatory T cells and the effector T cells, respectively, with respect to the total number of CD4⁺ T cells as determined by the FACS analysis of the mononulclear cell fraction immediately after isolation from the peripheral blood. CD62L^{high} CD25⁺CD4⁺ T cells and CD62L^{low} CD4⁺ T cells were used as regulatory T cells (Treg) and effector T cells (Teff), respectively, in the FACS analysis, as reported in Koyama K, Kagamu H, et al. Reciprocal CD4⁺ T-cell balance of effector CD62L^{low} CD4⁺ and CD62L^{high} CD25⁺ CD4⁺ regulatory T cells in small cell lung cancer reflects disease stage. Clin Cancer Res. 2008; 14: 6770-9. Detection of DDX3X-responding T cells was not possible in any of the healthy individuals (HV), small cell lung cancer patients with distal metastasis (SCLC-ED), and cured small cell lung cancer patients. However, the experiment found the presence of DDX3X-responding, specific IFN-γ-producing T cells in 5 of 12 small cell lung cancer (SCLC-LD) patients with no distal metastasis. This result indicates that the small cell lung cancer patients with DDX3X-specific T cells have desirable prognosis.

In the table, “Yes” means detection of DDX3X-specific T cells in blood, and “No” means no detection of DDX3X-specific T cells in blood.

TABLE 3

| | | DDX3X- | | |
|---|---|---|---|---|
| | | specific T | | |
| | Subject | cell | % Treg | % Teff |
| SCLC-LD | #1 | Yes | 2.83 | 30 |
| | #2 | Yes | 1.06 | 26 |
| | #3 | No | 3.88 | 8.8 |
| | #4 | No | 3.48 | 9.7 |
| | #5 | Yes | 4.88 | 30 |
| | #6 | No | 4.95 | |
| | #7 | No | 7.53 | |
| | #8 | No | 8.59 | |
| | #9 | No | | |
| | #10 | No | | |
| | #11 | Yes | | |
| | #12 | Yes | 4.23 | |
| SCLC-ED | #13 | No | 4.15 | 60 |
| | #14 | No | 3.47 | 5.3 |
| | #15 | No | 1.62 | 30 |
| | #16 | No | 6.49 | |
| | #17 | No | 4.03 | |
| | #18 | No | 1.82 | 12 |
| Cured | #19 | No | 2.36 | |
| SCLC | #20 | No | 4.84 | 16.6 |
| | #21 | No | 3.98 | 21 |
| | #22 | No | 1.92 | 13.4 |
| | #23 | No | | 27 |
| HV | #24 | No | 0.95 | 8.1 |
| | #25 | No | 1.56 | 8.7 |
| | #26 | No | 2.37 | |
| | #27 | No | 1.12 | 16.2 |
| | #28 | No | 1.87 | 21.2 |
| | #29 | No | 1.57 | |

### Example 11

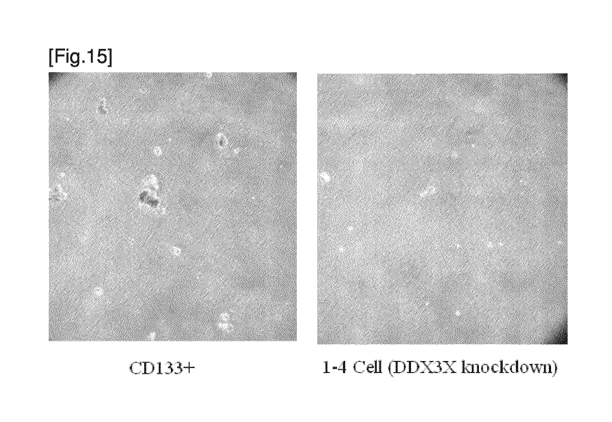

The 1-4 cells obtained by knockdown of DDX3X from the human colon cancer cell line HCT116 were examined for the ability to form floating cell aggregates (spheroids). In contrast to the parental strain HCT116 (CDD133+) that had the ability to form spheroids in a non-adherent culture, the DDX3X knockdown 1-4 cells did not have the spheroid forming ability (FIG. 15).

### Example 12

It is known that DDX3X has RNA helicase activities, and its involvement in the nucleo-cytoplasmic transport, processing, and maturation of miRNA in C. elegans and Drosophila is also known. However, there is no report of DDX3X involving in the miRNA of human cells. An investigation was thus conducted for the presence of miRNA fluctuations by knocking down DDX3X in human tumor cells and HCT116 with up-regulated DDX3X expression.

Experiments were conducted with 1-4 cells obtained by knockdown of DDX3X from the HCT116 cells, and mock-transfectant 1-6 cells, using a miRCURY LNAneration(trade name) microRNA Array 6th generation (Filgen). An miRBase Release17 was used as annotation information. GenePix 4000B (Molecular Devices) was used for array scans, and Array-Pro Analyzer Ver4.5 (Media Cybemetics) was used for creating image data and correcting image. Local regression was used for normalization. Of the 2,684 miRNAs analyzed, none had increased expression in 1-4 cells than in 1-6 cells. On the other hand, four miRNAs, hsa-miR-301a, hsa-miR-429, hsa-miR-301b, and hsa-miR-3922-3p were found that satisfied normalized intensity ≧10, normalized intensity (sum)≧negative control mean value (=85), and that had a reduced normalized intensity ratio ≧0.5 in 1-4 cells than in 1-6 cells.

1. A cancer malignancy evaluation method, comprising:
the step of measuring a DDX3X expression level in a cancer tissue; and
the step of evaluating malignancy of the cancer tissue by using the DDX3X expression level.

2. A cancer malignancy evaluation kit, comprising:
an antibody against DDX3X, or a polynucleotide that specifically binds to DDX3X mRNA or corresponding cDNA,
wherein the antibody or the polynucleotide is used for measurement of a DDX3X expression level in a cancer tissue.

3. A cancer prognosis evaluation method, comprising:
the step of detecting DDX3X-specific T cells in the blood of a cancer patient; and
the step of evaluating cancer prognosis by using the detection result.

4. A cancer prognosis evaluation kit, comprising:
DDX3X or a partial peptide thereof,
wherein the DDX3X or a partial peptide thereof is used for detection of DDX3X-specific T cells in the blood of a cancer patient.

5. A peptide consisting of:
a sequence of 9 to 20 contiguous amino acids that comprises the amino acid sequence represented by any of SEQ ID NOS: 2 to 87 in the amino acid sequence of SEQ ID NO: 1, or
an amino acid sequence essentially the same as such an amino acid sequence.

6. A peptide according to claim 5, wherein the peptide is a cancer antigenic peptide.

7. A cancer vaccine comprising the peptide of claim 5.

8. A cancer vaccine according to claim 7, wherein the vaccine is used for preventing or treating cancer, or for suppressing cancer metastasis or cancer recurrence.

9. An adoptive immunity cell producing method comprising the step of pulsing a cell having an antigen-presenting ability with DDX3X or a partial peptide thereof.

10. An antigen-presenting cell pulsed with DDX3X or a partial peptide thereof.

11. A DDX3X-specific T cell inducer comprising the antigen-presenting cell of claim 10 as an active component.

12. A method of producing an adoptive immunity cell composition comprising:
the step of exposing a cell having an antigen-presenting ability to DDX3X or a partial peptide thereof to obtain a cell presenting an antigen derived from the DDX3X or a partial peptide thereof; and
the step of inducing a DDX3X-specific T cell with the antigen-presenting cell.

13. A method according to claim 12, wherein the DDX3X-specific T cell is a DDX3X specific CD4-positive T cell.

14. A compound that inhibits DDX3X expression or activity for use in preventing or treating cancer, or for suppressing cancer metastasis or cancer recurrence.


SEQUENCE LISTING (97 sequences)

SEQ ID NO: 1 - protein, 662 aa, Homo sapiens
MSHVAVENALGLDQQFAGLDLNSSDNQSGGSTASKGRYIPPHLRNREATKGFYDKDSSGWSSSKDKDAYSSFGSRSDSRGKSSFFSDRGSGSRGRFDDRGRSDYDGIGSRGDRSGFGKFERGGNSRWCDKSDEDDWSKPLPPSERLEQELFSGGNTGINFEKYDDIPVEATGNNCPPHIESFSDVEMGEIIMGNIELTRYTRPTPVQKHAIPIIKEKRDLMACAQTGSGKTAAFLLPILSQIYSDGPGEALRAMKENGRYGRRKQYPISLVLAPTRELAVQIYEEARKFSYRSRVRPCVVYGGADIGQQIRDLERGCHLLVATPGRLVDMMERGKIGLDFCKYLVLDEADRMLDMGFEPQIRRIVEQDTMPPKGVRHTMMFSATFPKEIQMLARDFLDEYIFLAVGRVGSTSENITQKVVWVEESDKRSFLLDLLNATGKDSLTLVFVETKKGADSLEDFLYHEGYACTSIHGDRSQRDREEALHQFRSGKSPILVATAVAARGLDISNVKHVINFDLPSDIEEYVHRIGRTGRVGNLGLATSFFNERNINITKDLLDLLVEAKQEVPSWLENMAYEHHYKGSSRGRSKSSRFSGGFGARDYRQSSGASSSSFSSSRASSSRSGGGGHGSSRGFGGGGYGGFYNSDGYGGNYNSQGVDWWGN

SEQ ID NO: 2 - protein, 9 aa, Homo sapiens
FLLDLLNAT

SEQ ID NO: 3 - protein, 9 aa, Homo sapiens
NITQKVVWV

SEQ ID NO: 4 - protein, 9 aa, Homo sapiens
IQMLARDFL

SEQ ID NO: 5 - protein, 9 aa, Homo sapiens
TFPKEIQML

SEQ ID NO: 6 - protein, 9 aa, Homo sapiens
KYDDIPVEA

SEQ ID NO: 7 - protein, 9 aa, Homo sapiens
RYIPPHLRN

SEQ ID NO: 8 - protein, 9 aa, Homo sapiens
RNINITKDL

SEQ ID NO: 9 - protein, 9 aa, Homo sapiens
KQYPISLVL

SEQ ID NO: 10 - protein, 9 aa, Homo sapiens
IGLDFCKYL

SEQ ID NO: 11 - protein, 9 aa, Homo sapiens
IELTRYTRP

SEQ ID NO: 12 - protein, 9 aa, Homo sapiens
TRYTRPTPV

SEQ ID NO: 13 - protein, 9 aa, Homo sapiens
MGNIELTRY

SEQ ID NO: 14 - protein, 9 aa, Homo sapiens
LVLAPTREL

SEQ ID NO: 15 - protein, 9 aa, Homo sapiens
YPISLVLAP

SEQ ID NO: 16 - protein, 9 aa, Homo sapiens
QYPISLVLA

SEQ ID NO: 17 - protein, 10 aa, Homo sapiens
LEDFLYHEGY

SEQ ID NO: 18 - protein, 9 aa, Homo sapiens
FLDEYIFLA

SEQ ID NO: 19 - protein, 9 aa, Homo sapiens
LLVEAKQEV

SEQ ID NO: 20 - protein, 9 aa, Homo sapiens
FLLPILSQI

SEQ ID NO: 21 - protein, 9 aa, Homo sapiens
DFLDEYIFL

SEQ ID NO: 22 - protein, 9 aa, Homo sapiens
SHVAVENAL

SEQ ID NO: 23 - protein, 9 aa, Homo sapiens
VAVENALGL

SEQ ID NO: 24 - protein, 9 aa, Homo sapiens
ALGLDQQFA

SEQ ID NO: 25 - protein, 9 aa, Homo sapiens
LGLDQQFAG

SEQ ID NO: 26 - protein, 9 aa, Homo sapiens
GLDQQFAGL

SEQ ID NO: 27 - protein, 9 aa, Homo sapiens
DQQFAGLDL

SEQ ID NO: 28 - protein, 9 aa, Homo sapiens
NSSDNQSGG

SEQ ID NO: 29 - protein, 9 aa, Homo sapiens
KGRYIPPHL

SEQ ID NO: 30 - protein, 9 aa, Homo sapiens
PHLRNREAT

SEQ ID NO: 31 - protein, 9 aa, Homo sapiens
RGRGDYDGI

SEQ ID NO: 32 - protein, 9 aa, Homo sapiens
YDGIGSRGD

SEQ ID NO: 33 - protein, 9 aa, Homo sapiens
RSGFGKFER

SEQ ID NO: 34 - protein, 9 aa, Homo sapiens
KPLPPSERL

SEQ ID NO: 35 - protein, 9 aa, Homo sapiens
LFSGGNTGI

SEQ ID NO: 36 - protein, 9 aa, Homo sapiens
FSGGNTGIN

SEQ ID NO: 37 - protein, 9 aa, Homo sapiens
INFEKYDDI

SEQ ID NO: 38 - protein, 9 aa, Homo sapiens
YDDIPVEAT

SEQ ID NO: 39 - protein, 9 aa, Homo sapiens
TGNNCPPHI

SEQ ID NO: 40 - protein, 9 aa, Homo sapiens
EIIMGNIEL

SEQ ID NO: 41 - protein, 9 aa, Homo sapiens
IIMGNIELT

SEQ ID NO: 42 - protein, 9 aa, Homo sapiens
IPIIKEKRD

SEQ ID NO: 43 - protein, 9 aa, Homo sapiens
GSGKTAAFL

SEQ ID NO: 44 - protein, 9 aa, Homo sapiens
TAAFLLPIL

SEQ ID NO: 45 - protein, 9 aa, Homo sapiens
AAFLLPILS

SEQ ID NO: 46 - protein, 9 aa, Homo sapiens
IYADGPGEA

SEQ ID NO: 47 - protein, 9 aa, Homo sapiens
LAVQIYEEA

SEQ ID NO: 48 - protein, 9 aa, Homo sapiens
IYEEARKFS

SEQ ID NO: 49 - protein, 9 aa, Homo sapiens
RPCVVYGGA

SEQ ID NO: 50 - protein, 9 aa, Homo sapiens
CVVYGGADI

SEQ ID NO: 51 - protein, 9 aa, Homo sapiens
LLVATPGRL

SEQ ID NO: 52 - protein, 9 aa, Homo sapiens
ATPGRLVDM

SEQ ID NO: 53 - protein, 9 aa, Homo sapiens
GLDFCKYLV

SEQ ID NO: 54 - protein, 9 aa, Homo sapiens
LDFCKYLVL

SEQ ID NO: 55 - protein, 9 aa, Homo sapiens
LVLDEADRM

SEQ ID NO: 56 - protein, 9 aa, Homo sapiens
VLDEADRML

SEQ ID NO: 57 - protein, 9 aa, Homo sapiens
GFEPQIRRI

SEQ ID NO: 58 - protein, 9 aa, Homo sapiens
FSATFPKEI

SEQ ID NO: 59 - protein, 9 aa, Homo sapiens
YIFLAVGRV

SEQ ID NO: 60 - protein, 9 aa, Homo sapiens
RVGSTSENI

SEQ ID NO: 61 - protein, 9 aa, Homo sapiens
ATGKDSLTL

SEQ ID NO: 62 - protein, 9 aa, Homo sapiens
SLTLVFVET

SEQ ID NO: 63 - protein, 9 aa, Homo sapiens
FLYHEGYAC

SEQ ID NO: 64 - protein, 9 aa, Homo sapiens
LYHEGYACT

SEQ ID NO: 65 - protein, 9 aa, Homo sapiens
LHQFRSGKS

SEQ ID NO: 66 - protein, 9 aa, Homo sapiens
QFRSGKSPI

SEQ ID NO: 67 - protein, 9 aa, Homo sapiens
ILVATAVAA

SEQ ID NO: 68 - protein, 9 aa, Homo sapiens
TAVAARGLD

SEQ ID NO: 69 - protein, 9 aa, Homo sapiens
ISNVKHVIN

SEQ ID NO: 70 - protein, 9 aa, Homo sapiens
LPSDIEEYV

SEQ ID NO: 71 - protein, 9 aa, Homo sapiens
EYVHRIGRT

SEQ ID NO: 72 - protein, 9 aa, Homo sapiens
LGLATSFFN

SEQ ID NO: 73 - protein, 9 aa, Homo sapiens
TSFFNERNI

SEQ ID NO: 74 - protein, 9 aa, Homo sapiens
FFNERNINI

SEQ ID NO: 75 - protein, 9 aa, Homo sapiens
NITKDLLDL

SEQ ID NO: 76 - protein, 9 aa, Homo sapiens
DLLDLLVEA

SEQ ID NO: 77 - protein, 9 aa, Homo sapiens
EVPSWLENM

SEQ ID NO: 78 - protein, 9 aa, Homo sapiens
AYEHHYKGS

SEQ ID NO: 79 - protein, 9 aa, Homo sapiens
EHHYKGSSR

SEQ ID NO: 80 - protein, 9 aa, Homo sapiens
SRFSGGFGA

SEQ ID NO: 81 - protein, 9 aa, Homo sapiens
FGARDYRQS

SEQ ID NO: 82 - protein, 9 aa, Homo sapiens
GGGYGGFYN

SEQ ID NO: 83 - protein, 9 aa, Homo sapiens
GGYGGFYNS

SEQ ID NO: 84 - protein, 9 aa, Homo sapiens
GGFYNSDGY

SEQ ID NO: 85 - protein, 9 aa, Homo sapiens
SDGYGGNYN

SEQ ID NO: 86 - protein, 9 aa, Homo sapiens
GGNYNSQGV

SEQ ID NO: 87 - protein, 9 aa, Homo sapiens
NYNSQGVDW

SEQ ID NO: 88 - protein, 15 aa, Homo sapiens
KQYPISLVLAPTREL

SEQ ID NO: 89 - protein, 18 aa, Homo sapiens
EIIMGNIELTRYTRPTPV

SEQ ID NO: 90 - protein, 15 aa, Homo sapiens
KGADSLEDFLYHEGY

SEQ ID NO: 91 - protein, 20 aa, Homo sapiens
FVETKKGADSLEDFLYHEGY

SEQ ID NO: 92 - protein, 9 aa, Artificial Sequence
  peptide
XYPISLVLA

SEQ ID NO: 93 - RNA, 86 nt, Homo sapiens
acugcuaacgaaugcucugacuuuauugcacuacuguacuuuacagcuagcagugcaauaguauugucaaagcaucugaaagcagg

SEQ ID NO: 94 - RNA, 78 nt, Homo sapiens
gccgcaggugcucugacgagguugcacuacugugcucugagaagcagugcaaugauauugucaaagcaucugggacca

SEQ ID NO: 95 - RNA, 83 nt, Homo sapiens
cgccggccgaugggcgucuuaccagacaugguuagaccuggcccucugucuaauacugucugguaaaaccguccauccgcugc

SEQ ID NO: 96 - RNA, 84 nt, Homo sapiens
ggaagagucaagucaaggccagaggucccacagcagggcuggaaagcacaccugugggacuucuggccuugacuugacucuuuc

SEQ ID NO: 97 - DNA, 58 bp, Homo sapiens
ccggacgttctaagagcagtcgattctcgagaatcgactgctcttagaacgttttttg